(19) United States
(12) Patent Application Publication
(10) Pub. No.: US 2007/0092475 A1
(43) Pub. Date: Apr. 26, 2007

(54) Methods for enhancing the morphology, tone, texture and/or appearance of skin using a Meadowestolide

(76) Inventor: Alan Wohlman, Northbrook, IL (US)

Correspondence Address: Keith D. Parr;Lord, Bissell & Brook LLP, 115 S. La Salle Street, Chicago, IL, 60603, US

(21) Appl. No.: 11/256,722

(22) Filed: Oct. 24, 2005

(51) Int. Cl. A61K 8/37

(52) U.S. Cl. 424/70.22

(57) ABSTRACT

The present invention provides inexpensive, safe and reliable methods for improving the morphology, tone, texture and/or appearance of the skin of a mammal, preferably without using one or more surfactants (or other chemicals) that can strip away, or otherwise remove, one or more protective lipids of the skin. These methods comprise topically applying to the skin on a regular basis at least three applications of a composition in an effective amount including: (a) a Meadowestolide in an amount that is effective for enhancing the morphology, tone, texture and/or appearance of the skin, and having the chemical structure set forth herein; and (b) a cosmetically acceptable base in an amount that is effective for acting as a carrier vehicle for the Meadowestolide.

### BACKGROUND OF THE INVENTION

1. Field of the Invention

The present invention relates to methods for enhancing the morphology, tone, texture and/or appearance of the skin of mammals by topically administering, or otherwise applying, effective amounts of a Meadowestolide having the chemical structure set forth herein, as an active ingredient, thereto.

2. Background

The skin of mammals often becomes damaged or deteriorated as a result of sun exposure, exposure to chemicals, age, illness, abuse and/or similar factors. In order to eliminate or diminish lines, wrinkles, sun or age spots and/or sagging of the skin, many human beings apply a wide variety of skin care products to their skin, inject Botox (a highly purified preparation of a toxin produced by the bacterium Clostridium botulinum) into their skin and/or have plastic surgery (laser surgery, face lifts, eye lifts and the like). These treatments are often very expensive, and can present significant health risks to the humans, including death.

The water content of the skin stratum comeum (the outermost layer of the epidermis, or external skin surface) and surface lipids are important factors in the appearance and function of the skin. A disruption in the balance between the skin stratum comeum and surface lipids may lead to a clinical manifestation of dryness of skin.

The integrity of the skin depends upon the continued production and maintenance of structural and active biological materials, the configuration and function of which are largely dependent upon the local environment in which they exist. This environment can be defined, in part, by the quantitative balance of oil and water in the skin tissue. A primary challenge for those who strive to provide effective skin care treatments is a development of ingredients and formulations that facilitate a preservation of the correct balance of lipid and aqueous components in the complex layers that comprise the epidermal surface of the skin. A frequent use of surfactants on a mammal's skin often has a detrimental effect of stripping away, or otherwise reducing, a significant amount of protective lipids from the skin, which disadvantageously enables an evaporative loss of water and a consequent dehydration of the skin.

It would be beneficial to provide inexpensive, safe and reliable methods for enhancing the morphology, tone, texture and/or appearance of the skin of a mammal without using one or more surfactants (or similar chemicals), Botox or plastic surgery.

### Description of Related Art

Published U.S. Patent Application No. U.S. 2002/0086039 A1 describes skin and hair care products that include as a component bioactive glass (an inorganic glass material having an oxide or silicon as its major component) in a particulate form, in an aqueous solution or in a combination thereof. In contrast with the compositions employed in the methods of the present invention, and with such methods, this published patent application does not teach or suggest a use of an estolide, let alone a Meadowestolide having a chemical structure as described herein, a concentration of any type of estolide, let alone a concentration of a Meadowestolide having a chemical structure as described herein, or an application rate that includes more than one application of a product to skin. All of the teachings and examples that are present in this published patent application disclose only one application of a product described therein to skin. Further, the compositions employed in the methods of the present invention preferably do not include bioactive glass (in any form), or a composition derived from an aqueous extract of bioactive glass. While this published patent application describes meadowfoam seed oil (whole oil) as a common ingredient in lip liner products, the compositions that are employed in the methods of the invention need not include meadowfoam seed oil, or any components of meadowfoam seed oil.

Published U.S. Patent Application No. U.S. 2004/0156802A1 describes skin and hair cosmetics that comprise a chemical peeling agent, a bactericide, an anionic surfactant or a cationic surfactant and a cystine derivative, or a salt thereof, such as N,N′-diacetyl-L-cystine dimethyl ester, N-acetylcysteine or N-acetylhomocysteine. The cysteine derivative, which is stated to be capable of mitigating irritation or inflammation of the skin caused by the other components, is a required component of the cosmetics. In contrast with the compositions employed in the methods of the present invention, and with such methods, this published patent application does not teach or suggest an application of an estolide to skin, let alone a Meadowestolide having a chemical structure as described herein, a concentration of any type of estolide, let alone a concentration of a Meadowestolide having a chemical structure as described herein, or an application rate that includes more than one application of a product to skin. All of the teachings and examples that are present in this published patent application disclose only one application of a product described therein to skin. Further, the compositions employed in the methods of the present invention preferably do not include cystine, a cystine derivative, or a salt thereof.

Published U.S. Patent Application No. U.S. 2005/0129632 A1 describes UV absorber compositions that are stated to be suitable as UV filters in cosmetic and pharmaceutical compositions (i.e. to protect human and animal skin and hair from the harmful effects of UV radiation). The UV absorber compositions comprise: (a) from 1 to 99% by weight of a hydroxyphenyltriazine compound of a specified formula, and (b) from 99 to 1% by weight of a further UV absorber, which is selected from a specified group. In contrast with the compositions employed in the methods of the present invention, and with such methods, this published patent application does not teach or suggest an application of an estolide to skin, let alone a Meadowestolide having a chemical structure as described herein, a concentration of a Meadowestolide, let alone a concentration of a Meadowestolide having a chemical structure as described herein, or an application rate that includes more than one application of a product to skin. All of the teachings and examples that are present in this published patent application disclose only one application of a product described therein to skin. Further, the compositions employed in the methods of the present invention preferably do not include a hydroxyphenyltriazine compound, such as those described in the published patent application, or a HDI/trimethylol hexyl-lactone crosspolymer.

Published U.S. Patent Applications Nos. 2005/0069515 and 2005/0069517 describe compositions for application to the hair that may include meadowfoam seed oil (whole oil) and derivatives thereof, such as Meadowestolide. These published patent applications do not mention skin.

U.S. Pat. No. 5,380,894 describes a process for the production of hydroxy fatty acids and estolide intermediate compounds, which may be used as lubricants, greases, plasticizers and printing inks, and in cosmetics, and the estolide intermediates produced by such process. The estolides are prepared by a reaction of one or more unsaturated fatty acids, which are naturally occurring in plant oils, in the presence of a suitable catalyst and water under suitable temperature and pressure conditions (about 230-250° C. and 30-60 psi). Following the reaction, a crude estolide fraction is separated from the catalyst by filtration, and water is removed. The estolides may be further purified by distillation. Meadowfoam oil, having a high content of Δ⁵ unsaturated fatty acids, is stated to be preferred as a source of the starting material. Example 1 describes the production of estolides from meadowfoam oil. Table I provides a summary of the starting fatty acids that are employed, and the estolides that are formed from the various fatty acids. In contrast with the methods of the present invention, and with the compositions employed therein, the '894 patent does not teach or suggest any information regarding how the estolides may be used in cosmetics (or otherwise). For example, the '894 patent does not teach or suggest any concentrations of estolides for use in cosmetics, any information regarding how estolides can be used in cosmetics or the types of cosmetics in which estolides can be employed, any application rates of estolides to skin, any amounts of estolides that may be applied to the skin, any effects that the estolides may have on the skin, or the like.

U.S. Pat. No. 6,051,214 describes shampoos and conditioners that include aqueous compositions of a surfactant cleansing agent and a thickener, or a hair conditioning agent, respectively, in combination with one or more fatty acid estolides. In contrast with the methods of the present invention, this patent does not discuss skin, an application of estolides to skin, the concentrations of Meadowestolide described herein for application to skin or the application rates described herein.

### SUMMARY OF THE INVENTION

It has been surprisingly and unexpectedly determined that an estolide derived from meadowfoam (Meadowestolide) having the particular chemical structure set forth hereinbelow is particularly effective for enhancing a morphology, tone, texture and/or appearance of a mammal's skin when employed in the manner, and under the conditions, described herein. The molecular configuration of this estolide has been found to be very dynamic, resulting in its ability to actively participate in tissue rehydration and conditioning of skin. Microphotographic images of the skin of human beings that are present herein provide a clear observable visualization of the improvement that occurs in the morphology, tone, texture and/or appearance of the skin when the skin is treated with Meadowestolide in the manner described herein (preferably contained in a cream emulsion base at a level of about 2 to about 3 weight percent) in comparison with untreated skin, or with skin that has been treated with various control preparations.

The methods of the present invention advantageously provide inexpensive, safe and reliable methods for enhancing the morphology, tone, texture and/or appearance of the skin of a mammal without the need for using one or more surfactants (or similar chemicals), Botox or plastic surgery.

In one aspect, the present invention provides a method for enhancing a morphology, tone, texture and/or appearance of a mammal's skin comprising topically applying to the skin on a regular basis at least three applications of a composition including:
(a) a Meadowestolide in an amount that is effective for enhancing the morphology, tone, texture and/or appearance of the skin, wherein the Meadowestolide has the structure: 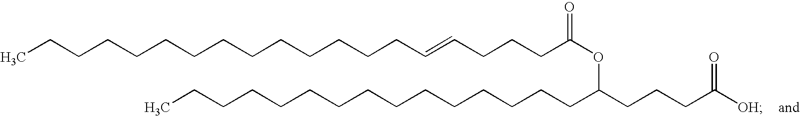
(b) a cosmetically acceptable base in an amount that is effective for acting as a carrier vehicle for the Meadowestolide;
   wherein the amount of the composition that is applied to the skin is an amount that is effective for enhancing the morphology, tone, texture and/or appearance of the mammal's skin;
   wherein the pH of the composition ranges from about 2 to about 11; and
   wherein the morphology, tone, texture and/or appearance of the mammal's skin is enhanced.

In another aspect, the present invention provides a method for enhancing a morphology, tone, texture and/or appearance of a mammal's skin comprising topically applying to the skin on a regular basis at least three applications of a composition consisting essentially of:
(a) a Meadowestolide in an amount that is effective for enhancing the morphology, tone, texture and/or appearance of the skin, wherein the Meadowestolide has the structure: 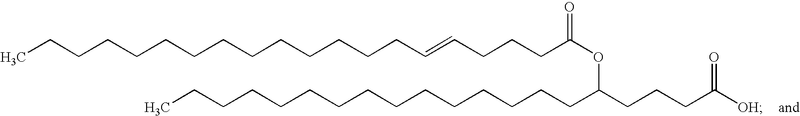
(b) a cosmetically acceptable base in an amount that is effective for acting as a carrier vehicle for the Meadowestolide;
   wherein the amount of the composition that is applied to the skin is an amount that is effective for enhancing the morphology, tone, texture and/or appearance of the mammal's skin;
   wherein the pH of the composition ranges from about 2 to about 11; and
   wherein the morphology, tone, texture and/or appearance of the mammal's skin is enhanced.

It is preferred that a Meadowestolide having the chemical structure set forth herein is the only active agent (compound or substance that has an ability to produce an observable or otherwise detectable effect upon a mammal's skin) that is employed in the compositions that are used in the methods of the present invention.

The compositions that are employed in the methods of the invention, and such methods, are safe for use by mammals and typically do not irritate the mammals' skin.

### BRIEF DESCRIPTION OF THE DRAWINGS

The patent or application file contains at least one drawing and/or photograph executed in color. Copies of this patent or patent application publication with color drawing(s) and/or photograph(s) will be provided by the Office upon request and payment of the necessary fee.

FIG. 1 is a photograph showing the water retention results of the in vitro water binding experiments that were performed with Meadowestolide, Meadowfoam seed oil, a Meadowfoam seed oil fatty acid and Jojoba oil, as is described in Example 3.

FIG. 2 is a graph showing the water retention results of the in vitro water binding experiments that were performed with Meadowestolide, Meadowfoam seed oil, a Meadowfoam seed oil fatty acid and Jojoba oil, as is described in Example 3.

FIG. 3 is a set of two graphs showing the results of the in vivo transepidermal water loss (TEWL) and tissue hydration experiments, respectively, that were performed with Meadowestolide, Meadowfoam seed oil and a long-chain guerbet fatty ester, as is described in Example 4.

FIG. 4 is a series of four Micro-Topological Epidermal Imaging photographs produced using Micro-Topological Epidermal Imaging (MTEI) and showing the skin of a 22 year old female subject after a Meadowestolide skin cream had been applied to the right side of her face, and a control had been applied to the left side of her face, each once per day for a period of five days, as is described in Example 5.

FIG. 5 is a series of four Micro-Topological Epidermal Imaging photographs produced using Micro-Topological Epidermal Imaging (MTEI) of the hands of a 63-year old male subject, as is described in Example 5. The upper two photographs show both of the hands prior to an application of any product. The lower right photograph shows one of the hands after ten applications of a Meadowestolide skin cream base containing 3 weight percent Meadowestolide (five times per day over a period of two days). The lower left photograph shows the other hand after ten applications of a control skin cream base containing 3 weight percent of glycerine in place of the Meadowestolide (five times per day over a period of two days).

FIG. 6 is a series of four Micro-Topological Epidermal Imaging photographs produced using Micro-Topological Epidermal Imaging (MTEI) of the hands of a 63-year old male subject, also as is described in Example 5. FIG. 6 shows the same hands as those shown in the lower two photographs of FIG. 5. The upper right photograph shows one of the hands at a magnification of 20× after ten applications of a Meadowestolide skin cream product containing 3 weight percent Meadowestolide (five times per day over a period of two days). The lower right photograph shows the same hand as in the upper right photograph, with the exception that the magnification is 100×. The upper left photograph shows one of the hands at a magnification of 20× after ten applications of a control skin cream base containing 3 weight percent of glycerine in place of the Meadowestolide (five times per day over a period of two days). The lower left photograph shows the same hand as in the upper left photograph, with the exception that the magnification is 100×.

### DETAILED DESCRIPTION OF THE PREFERRED EMBODIMENTS

The present invention may be understood more readily by reference to the following detailed description of the preferred embodiments of the invention, and to the examples included therein.

Definitions

For purposes of clarity, various terms and phrases used throughout this specification and the appended claims are defined in the manner set forth below. If a term or phrase used in this specification, or in the appended claims, is not defined below, or otherwise in this specification, the term or phrase should be given its ordinary meaning.

The phrase “antioxidant agent” as used herein means an agent that has an ability to prevent, reduce or aid in the prevention or reduction of, an oxidation, degradation and/or other decomposition of one or more ingredients or components, such as a fat or an oil, and/or to prevent, or aid in the prevention of, oxygen-based damage to hair, skin or other cells. Antioxidant agents include, for example, various tocopherol mixtures, edible acids (citric acid, ascorbic acid and the like), vitamin A, vitamin C, vitamin E, beta-carotene, selenium, magnesium, herbal extracts, such as a Rosemary, Sage, Oregano, Ginger, Marjoram or Rosemary Oleoresins extract, plant phenols, such as Vanillin, ellagic acid and Resveratrol, and synthetic antioxidants, such as tertiary butylhydroquinone (TBHQ), butylated hydroxyamisole (BHA) or butylated hydroxytoluene (BHT), or mixtures thereof.

The phrase “carrier vehicle” as used herein means a medium for providing a controlled delivery of an active agent to a mammal, or part thereof, such as the skin.

The term “component” as used herein means a part, portion, element, constituent or ingredient, and is used interchangeably with “ingredient.” For example, in connection with a composition employed in the methods of the invention, this term means an ingredient, or combination of ingredients, used in the composition, or a part, portion, element or constituent thereof, depending upon the context in which this term is used, which may readily be determined by those of skill in the art.

The phrase “cosmetically acceptable base” as used herein means a base that can serve as a carrier vehicle for a Meadowestolide having the chemical structure described herein, and that may be applied to the skin of a normal and healthy mammal in a reasonable quantity over a reasonable period of time, preferably without an unreasonable risk of harm, injury, inflammation, irritation or allergic response to, or by, the skin, or illness or harm to the mammal. Persons of ordinary skill in the art may readily determine whether or not a particular base is a cosmetically acceptable base.

The phrase “inexpensive” as used herein means relatively inexpensive in comparison with other methods that attempt to achieve the same or similar results.

The phrase “diluent” as used herein means a substance or agent that dilutes, facilitates a physical separation of one or more ingredients and/or makes thinner or weaker. Diluent materials that are suitable for use with mammals could generally include complex polysaccharides, carbohydrates, smaller sugars (dextrose, sucrose and the like), dicalcium phosphate, tricalcium phosphate, maltodextrin and water.

The phrase “does not irritate the mammal's skin” as used herein means that a composition employed in the methods of the invention, and such methods, does not result in a significant amount of detectable irritation (redness, burning, itching, adverse change in skin surface, inflammation and/or chafing) to the skin of an average, healthy mammal that does not have, or suffer from, or have a history or having, or suffering from, any significant (or any) skin allergies or diseases (including allergic hypersensitivity). Skin irritation and allergic hypersensitivity may be determined using standard methods and equipment known by those of skill in the art.

The term “emulsifier” as used herein means any substance or agent that aids in the formation of an emulsion, such as egg yolk, egg lecithin, soy lecithin and mono- and di-glycerides.

The term “emulsion” as used herein means a generally stable and homogeneous mixture of two liquids that do not normally mix (i.e., they are immiscible between themselves), such as vegetable oil and water, for example, milk and mayonnaise. Emulsions can be true colloids or less stable mixtures, such as salad dressing, which tend to separate in a short time. An emulsion can often be broken down (i.e. the liquids separated) by factors such as mechanical manipulation, chemical effects and/or time.

The phrase “enhancing the morphology, tone, texture and/or appearance of the skin of mammals” as used herein means having an observable or otherwise detectable improvement in the morphology, tone, texture and/or appearance of the skin of a mammal, for example, an elimination or reduction in the quantity and/or severity of skin lines, wrinkles, sagging, age and/or sun spots, bumps, blisters, dryness, redness (or other discoloration) or other defects or deformations, an increase in the taughtness/tightness or smoothness of the skin or an increase in a youthful appearance of the skin. An enhancement in the morphology, tone, texture and/or appearance of the skin of a mammal may be detected visually, manually (by touch or feel) or using techniques known by those of skill in the art, by comparing the skin as it existed prior to applications of a composition thereto with the same skin as it exists after applications of a composition thereto.

The term “estolide” as used herein means a group of oligomeric fatty acids having an acyl backbone of a first fatty acid that contains a secondary ester linkage to a second fatty acid. They are ester-linked branched-chain polymeric molecules that are produced from fatty acids. They are generally partially unsaturated, and include mono- and poly-estolides.

The term “fat” as used herein means any of the various saturated and/or unsaturated (including monounsaturated and polyunsaturated), hydrogenated or unhydrogenated soft solid, semisolid and/or solid organic compounds that generally comprise the glyceride esters of fatty acids and associated phosphatides, sterols, alcohols, hydrocarbons, ketones and/or related compounds, components thereof and/or mixtures or other combinations thereof. Such components include, but are not limited to, fatty acids, glycerides (mono-, di- and tri-), ethyl and other esters of fatty acids, as well as components thereof, and combinations thereof. Fats occur widely in organic tissue, particularly in the subcutaneous connective tissue of animals (beef, poultry, pork, lamb, liver and the like), and in the seeds, nuts and fruits of plants. There is generally no chemical difference between fats and oils, with the only distinction being that fats are generally solid at room temperature and oils are generally liquid at room temperature. The phrase “fatty acids” as used herein means carboxylic acids that generally are derived from, or contained in, an animal, vegetable or other fat or oil, whether saturated, unsaturated, monounsaturated, polyunsaturated, aromatic, essential, nonessential, in a cis or trans form, in the ethyl esters, mono-, di- or tri-glycerides, free fatty acids or other forms, and components and combinations of the foregoing.

The phrase “fatty acids” as used herein means carboxylic acids that generally are derived from, or contained in, an animal, vegetable or other fat or oil, whether saturated, unsaturated, monounsaturated, polyunsaturated, aromatic, essential, nonessential, in a cis or trans form, in the ethyl esters, mono-, di- or tri-glycerides, free fatty acids or other forms, and components and combinations of the foregoing. Fatty acids include, but are not limited to, the specific fatty acids identified below:

| | Number of | Number of |
|---|---|---|
| Common Name | Carbon Atoms | Double Bonds |
| Butyric Acid | 4 | 0 |
| Caproic Acid | 6 | 0 |
| Caprylic Acid | 8 | 0 |
| Capric Acid | 10 | 0 |
| Lauric Acid | 12 | 0 |
| Myristic Acid | 14 | 0 |
| Palmitic Acid | 16 | 0 |
| Palmitoleic Acid | 16 | 1 |
| Stearic Acid | 18 | 0 |
| Oleic Acid | 18 | 1 |
| Linoleic Acid | 18 | 2 |
| Alpha-Linolenic Acid | 18 | 3 |
| (ALA) | | |
| Gamma-Linolenic Acid | 18 | 3 |
| (GLA) | | |
| Arachidic Acid | 20 | 0 |
| Gadoleic Acid | 20 | 1 |
| Arachidonic Acid (AA) | 20 | 4 |
| Eicosapentaenoic Acid | 20 | 5 |
| (EPA) | | |
| Behenic Acid | 22 | 0 |
| Erucic Acid | 22 | 1 |
| Docosahexaenoic Acid | 22 | 6 |
| Lignoceric Acid | 24 | 0 |

Other fatty acids are known by those of skill in the art. A wide variety of fatty acids are commercially available from sources known by those of skill in the art. Also, oils can be separated into their component fatty acids on a capillary column in a gas chromatograph, and the relative fatty acid contents measures. Additional information concerning fatty acids is readily available from the Fatty Acid Producer's Council (New York, N.Y.).

The term “humans” as used herein, unless otherwise stated, includes human beings that are babies, infants, children or adults.

The term “ingredient” is used herein interchangeably with “component” in connection with compositions described herein.

The abbreviation “INCI” as used herein means International Nomenclature Cosmetic Ingredient.

The phrase “leave-on” means a composition or product that is applied to the skin without a deliberate rinsing step within a specified period of time after its application to the skin.

The term “lipid” as used herein means any of a group of organic compounds, including fats, oils, waxes, sterols, and triglycerides, that generally are insoluble in water but soluble in nonpolar organic solvents, are oily to the touch, and together with carbohydrates and proteins constitute the principal structural material of living cells.

The phrase “liquid” as used herein means a fluid or semi-fluid, the shape of which is generally determined by the container that it fills.

The term “mammals” as used herein includes humans and non-human mammals.

The phrase “marine oil” as used herein includes, but is not limited to, “fish oil” and one or more individual components of marine oil, such as an omega-3 fatty acid, or a combination thereof. Marine oils include, for example, herring oil, cod oil, anchovy oil, tuna oil, sardine oil, menhaden oil and algae oil.

The terms “Meadowestolide” and “meadowfoam estolide” as used herein mean an estolide derived from meadowfoam, such as from an unsaturated fatty acid that is present in meadowfoam seed oil. For example the Δ⁵ monoene acids in meadowfoam seed oil form estolides under mild acid catalysis, with neighboring group participation by the carboxyl group facilitating the reaction.

The term “oil” as used herein means a fat that generally is viscous, liquid or liquefiable at room temperature, and includes mixtures and other combinations of one or more oils and/or components of oils, such as fatty acids, glycerides and/or ethyl esters of fatty acids (or components thereof).

The phrase “on a regular basis” as used herein means that a composition employed in the methods of the invention is applied to the skin of a mammal on a reasonably continuous basis (i.e. without delaying one or more applications for an unreasonably lengthy period of time), for example, a regular application of the composition to the mammal's skin one, two, three, four, five, six, seven, eight, nine, ten and so forth times within a period of one, two, three, four, five, six, seven, eight, nine, ten and so forth days for a duration of one, two, three, four, five, six, seven, eight, nine, ten and so forth days or weeks.

The phrase “plant seed oil” as used herein means an oil that is extracted, or otherwise obtained from, a seed of a plant, either directly or indirectly, particularly oily seeds, including one or more individual components thereof and mixtures thereof. Plant seed oils include, but are not limited to, Black Currant seed oil, Borage seed oil, safflower seed oil, sunflower seed oil, sesame seed oil, avocado seed oil, pumpkin seed oil, olive seed oil, coconut seed oil, rapeseed oil, flaxseed (linseed) oil, cottonseed oil, tung oil, meadowfoam seed oil, parsley seed oil, carrot seed oil, fennel fruit oil, parsnip seed oil, coriander seed oil, chervil seed oil, caraway plant oil and celery seed oil. Other plant seed oils are known by those of skill in the art.

The phrase “plant oil” as used herein means an oil that is extracted, or otherwise obtained from, a plant, either directly or indirectly, particularly oily plants, including one or more individual components thereof and mixtures thereof. Plant oils include, but are not limited to, Evening Primrose oil, Borage oil, safflower oil, sunflower oil, peanut oil, walnut oil, almond oil, avocado oil, olive oil, corn oil, soy oil, soybean oil, coconut oil, palm oil, palm kernel oil and castor oil. Other plant oils are known by those of skill in the art.

The term “plurality” as used herein means more than one, such as two, three, four, five, six, seven, eight, nine, ten, fifteen, twenty or the like.

The abbreviation “q.s.” as used herein means a sufficient quantity, for example, to cause the weight percent of a composition to be 100%, or to obtain a desired effect or benefit.

The phrase “rinse-off” means a composition or product that is applied to the skin with a deliberate rinsing step within a specified period of time after its application to the skin.

The phrase “room temperature” as used herein means the temperature in a room, which generally ranges from about 15° C. to about 30° C. (from about 59° F. to about 86° F.), and more usually ranges from about 21° C. to about 23° C. (from about 70° F. to about 74° F.). The “ambient temperature” of a room is “room temperature.” The phrase “safe for use” in connection with compositions described herein, and methods of the invention, means that the compositions, and the components contained therein, and the methods, using reasonable quantities of active and other components, and administered for reasonable periods of time (such as those quantities and periods of time described herein, or as otherwise recommended for a particular mammal by a physician, veterinarian or other skilled clinician), which may vary for different types of mammals, do not cause, or present an unreasonable risk of harm, damage, defect, disorder, deformity or injury to, or by, the skin of an average, healthy mammal that does not have, or suffer from, or have a history or having, or suffering from, skin allergies or diseases, or illness, disease or harm to an average, healthy mammal that does not have, or suffer from, or have a history or having, or suffering from, skin allergies or diseases. Preferably, such compositions and methods do not cause, or present, virtually any (or any) such risk.

The term “skin” as used herein in connection with human beings means the outer integument or covering of the body, generally consisting of the cornium, or dermis, and the epidermis, for example, the outer covering of the face (typically not including the lips) or the outer covering of the neck. In connection with non-human mammals, the term “skin” means the outer covering of the body that is not fur.

The phrases “surfactant” and “wetting agents” as used herein mean substances or agents that lower the surface tension (tendency of a liquid to reduce its exposed surface to the smallest possible area) of a liquid, generally allowing easier spreading, and/or the interfacial tension between two liquids. Surfactants are usually organic compounds that are amphipathic in that they contain both hydrophobic groups (their “tails”) and hydrophilic groups (their “heads”). Therefore, they are typically sparingly soluble in both organic solvents and water. Surfactants generally reduce the surface tension of water by adsorbing at the air-water interface, and reduce the interfacial tension between oil and water by adsorbing at the liquid-liquid interface. Many surfactants can also assemble in the bulk solution into aggregates that are known as micelles.

The phrases “topical administration” and “topically applying” as used herein mean an application onto the skin (generally onto one or more exposed or outer surfaces thereof, such as the epidermis), for example, using hands, fingers or a wide variety of applicators (spray, pump, brush, mat, powder puff, cotton ball, cue tip and the like). The application may be made, for example, by laying, pouring, spreading, spraying, sprinkling or massaging into, or onto, the skin or hair, or by any other convenient or suitable method.

As used herein, the term “viscosity” means resistance to flow (of a fluid or semi-fluid). Viscosity can be measured using, for example, a commercially available viscometer.

The term “wax” as used herein means a fatty substance that generally is solid at room temperature and softens and melts when warmed. Generally, waxes are similar in composition to fats and oils, with the exception that they do not contain glycerides. Some waxes are hydrocarbons, and others are esters of fatty acids and alcohols. Examples of waxes include, but are not limited to, beeswax, lanolin, carnauba, candelilla, ozokerite, bayberry, sugar cane, paraffin, microcrystalline and sorbitol.

General Description and Utility

The present invention provides cost-effective methods that are useful for enhancing the morphology, tone, texture and/or appearance of the skin, preferably without using any surfactants or other chemicals that can strip away one or more protective lipids of the skin, and that are safe for use by mammals. Such methods can be employed by skin care professionals, such as dermatologists, as well as by individuals, to enhance the morphology, tone, texture and/or appearance of the skin.

Meadowestolide

Meadowfoam, or Limnanthes alba, is a white flowered, herbaceous plant which gets its name because of its resemblance to a meadow of foam when blooming. The naturally-occurring oil that can be extracted from the seeds of meadowfoam (known as “meadowfoam oil” or “meadowfoam seed oil”), using techniques known by those of skill in the art, contains over 98% of long-chain fatty acids, predominantly as triglycerides. Meadowfoam seed oil has a high content of Δ⁵ unsaturated fatty acids (total fatty acids composed of approximately 60% 5-eicosenoic acid, 10% 5-docosenoic acid, 19% 5,13-docosadienoic acid, and less than 5% 18:1 Δ⁵ fatty acids). Meadowfoam seed oil is commercially available, for example, from The Fanning Corporation (Chicago, Ill.) and Botagenics, Inc. (Northridge, Calif.).

Meadowfoam seed oil estolides are one class of hundreds of different types of compounds or derivatives that may be obtained, or derived, from meadowfoam seed oil. The meadowfoam seed oil estolide (Meadowestolide) having the chemical structure described herein is one of hundreds of different types of meadowfoam seed oil estolides that may be derived from meadowfoam seed oil.

The Meadowestolide that is employed in the methods of the invention is a spatially oriented compound that can aid in reestablishing a physiologically correct balance of oil and water in epidermal tissue and, thereby, restore skin to a healthy, youthful morphology. This Meadowestolide, when applied to skin in reasonably low concentrations, such as in a base including from about 1 to about 5 weight percent of Meadowestolide, generally produces a marked sensory improvement in both the feel and appearance of the skin, and in the skin tone and texture. It has been surprisingly and unexpectedly determined as a result of a significant amount of experimentation that the methods of the present invention, which employ compositions that include a Meadowestolide having a particular chemical structure, achieve superior results in comparison with the same or similar methods that include meadowfoam oil (crude or refined).

The Meadowestolide that is employed in the methods of the present invention is commercially available from The Fanning Corporation (Chicago, Ill.). Alternatively, the Meadowestolide can be produced from meadowfoam seed oil using methods and equipment known by those of skill in the art, such as an acid catalyzed condensation of meadowfoam seed oil fatty acids to produce a dimeric ester product. Isbell and Kleiman, for example, have described a process for producing high yields of estolides from unsaturated fatty acids by acid catalysis (J. Amer. Oil. Chem. Soc. 71(4), 379-383, 1994). The Meadowestolide results from an acid catalyzed condensation of meadowfoam fatty acids to form a dimeric ester. Processes for producing estolides by various routes are also described in U.S. Pat. No. 5,380,894, Isbell et al., J. Amer. Oil. Chem. Soc. 71(1), 169-174 (1994) and Erhan et al., J. Amer. Oil. Chem. Soc. 74(3), 249-254 (1997) and Isbell et al., J. Amer. Oil. Chem. Soc. 74(4), 473-476 (1997).

Generally, methods for producing meadowfoam seed oil estolides include the steps of: (1) crushing (preferably to a flaked condition) the seeds of the plant Limnanthes alba; (2) washing the seeds with a solvent, whereby the oil from the seed becomes dissolved in the solvent; (3) separating the spent seeds and the solvent solution; (4) treating the solvent solution, for example, using distillation techniques, to remove the solvent from the oil extract: (5) deodorizing the oil extract; (6) hydrolyzing the oil extract to obtain free fatty acids present in the oil extract; (7) distilling the fatty acids; (8) reacting the fatty acids to estolides; and (9) recovering estolides. All of the foregoing steps may be performed using conventional techniques and equipment. Hydrolysis of the oils to the fatty acids may be achieved, for example, using conventional splitting techniques, alkali splitting of fats or splitting with steam under pressure. Suitable alkali splitting techniques include, for example, treatment with sodium methoxide or sodium or potassium hydroxide. See, for example, published U.S. Patent Application No. 2005/0042347 A1, which describes a method for producing an oil extract from the seeds of meadowfoam plants, U.S. Pat. Nos. 5,849,935 and 6,022,982, and “A.O.C.S. Tentative Method Ca-6b-53” in Official and Tentative Methods of the American Oil Chemist's Society, third edition, AOCS, Chicago, Ill. (1973). Additionally, U.S. Pat. No. No. 5,380,894 describes a process for the production of estolides from unsaturated fatty acids in the presence of water and a clay catalyst.

It is preferred that the Meadowestolide that is employed in the methods of the invention is employed in a substantially pure form, and that no other components of, or compounds contained in, meadowfoam oil (crude or refined), and no meadowfoam oil, are employed in these methods, or are included in the compositions that are employed in these methods. Methods for purifying Meadowestolide are known by those of skill in the art. It is also preferred that no active agents, substances or compounds (agents, substances or compounds that, in large enough quantities, have an ability to produce an effect or change to the morphology, tone, texture and/or appearance of the skin), from any source, other than Meadowestolide are included in the compositions that are employed in the methods of the invention.

The total amount of the Meadowestolide that is effective for enhancing the morphology, tone, texture and/or appearance of the skin may vary widely, depending upon a variety of factors, such as the type, age, sex, genetic predisposition and general health of the mammal, as well as the mammal's sun exposure, and the type and formulation of the particular base being employed, and may readily be determined by those of skill in the art. Typically, a total amount of from about 0.10 to about 20 weight percent, based upon the total weight of the compositions, of the Meadowestolide is present in the compositions that are employed in the methods of the invention. For example, the weight percent of the Meadowestolide may be about one, two, three, four, five, six, seven, eight, nine, ten, eleven, twelve, thirteen, fourteen, fifteen, sixteen, seventeen, eighteen, nineteen or twenty. The amount of the Meadowestolide preferably ranges from about 1 to about 5 weight percent, and more preferably ranges from about 1.5 to about 4.5 weight percent, and even more preferably ranges from about 2.0 to about 4.0 weight percent, and most preferably ranges from about 2.5 to about 3.5 weight percent. The amount of the Meadowestolide, in some cases, may range from about 0.10 to about 4 weight percent and, in other cases, may range from about 6 to about 20 weight percent.

Cosmetically Acceptable Base

A wide variety of cosmetically acceptable bases, which may be employed as carrier vehicles, diluents or dispersants for the active ingredient (Meadowestolide) that is employed in the compositions that are used in the methods of the invention, are known by those of skill in the art, and are commercially available from sources known by those of skill in the art, such as The Fanning Corporation (Chicago, Ill.). For example, a variety of cosmetically acceptable bases are described in “Cosmetic Additives—An Industrial Guide” (William Andrew Publishing, 1991), “Cosmetic and Toiletry Formulations” (Volume 3, 2^{nd} Edition, William Andrew Publishing, 1995), “Handbook of Cosmetics and Personal Care” (Gower Publishing Unlimited, ISBN 0566074702, 1994), “Cosmetic Ingredients” (Three Rivers Press, ISBN 0609803670, 1999) and “Cosmetics Unmasked” (Thorsons, Harper Collins, ISBN 0-00-710568-1, 2001), or are commercially available from Base Formula, Ltd. (Melton Mowbray, England), Well Naturally Products, Ltd. (Blaine, Wash.), Essential Wholesale (Clackamas, Oreg.), Urist Cosmetics, Inc. (Richmond, Calif.) and Sciencelab.com, Inc. (Houston, Tex.).

Ingredients that may generally be included in and/or used to produce a cosmetically acceptable base include, but are not limited to, any suitable combination and amounts of cosmetically acceptable ingredients, which may readily be determined by those of skill in the art, such as the following, as well as those that are described in the Examples set forth hereinbelow:

Glycols
Glycerin
Propylene Glycol
Butylene Glycol
Hexylene Glycol
2-Methyl Propane Diol
Alcohols
Ethanol
Isopropanol
n-propanol
lauryl alcohol
oleyl alcohol
Esters
Isopropyl Myristate
Isopropyl Palmitate
Jojoba Oil
Glyceryl tri caprate/caprylate
Butyl Acetate
Propylene Glycol di Caprate/Caprylate
Sorbitan Esters
Diesters of Diacids
Ethyl Acetate
Ethoxylated Materials
Ethoxylated Fatty Alcohols
Ethoxylated Fatty Acids
Ethoxylated Sorbitan Esters
Ethoxylated Glycerides
Ethoxydiglycol
Aerosol Propellants
Propane
Butane
Pentane
Isobutane
HFC, CFC, HCFC
Waxes/Bases
Lubragels
Zigels
Jojoba Glaze
Absorption Bases
Ketones
Acetone
Methyl Ethyl Ketone
Propoxylated Materials
Propoxylated Fatty Alcohols
Propoxylated Fatty Acids
Esters of Propoxylated Fatty Alcohols
Ethoxylated Propoxylates
Aerosol Propellant Gases
Anhydrous Ionic Surfactants
Phosphate Esters
Sulfates
Carboxylates
Fatty Amine Salts
Quaternary Nitrogen Salts
Fats, Oils and Waxes
Derived from Animals, Plants, Seeds,
Minerals, Marine or Other Sources
Silicones
Dimethicone
Simethicone
Cyclomethicone
Dimethicone Ethoxylates and
and Propoxylates
Fluorocarbons and Derivatives
Zonyls
Fluorocarbon Alcohols
Amides
Fatty Acid Diethanolamides
Fatty Acid Monoethanolamides
Fatty Acid Dimethylaminopropyl
Amides
Polymers
Polyalkenes
Polyoxethylenes
Polyoxypropylenes
Polyamides
Polyesters
Polyurethanes
Cellulostics and Derivatives
Copolymers
Aliphatic Compounds
n-alkanes
branched alkanes
Permethyls

Methods, equipment, techniques and ingredients that are generally employed in the preparation of skin care products or components, such as cosmetically acceptable bases, are well known by those of skill in the art.

The amount of a cosmetically acceptable base that is included in the compositions that are employed in the methods of the invention is an amount that is effective for acting as a carrier vehicle, diluent or dispersant for the Meadowestolide. Such amount will generally be 100 weight percent (total weight of the composition) minus the total weight percent of the Meadowestolide and typically ranges from about 80 to about 99.9 weight percent, based upon the total weight of the compositions. For example, the weight percent of the base may be about eighty, eighty-one, eighty-two, eighty-three, eighty-four, eighty-five, eighty-six, eighty-seven, eighty-eight, eighty-nine, ninety, ninety-one, ninety-two, ninety-three, ninety-four, ninety-five, ninety-six, ninety-seven, ninety-eight or ninety-nine. Such an amount preferably ranges from about 95 to about 99 weight percent, more preferably ranges from about 95.5 to about 98.5 weight percent and most preferably ranges from about 96.5 to about 97.5 weight percent. The amounts of the various ingredients that may be present in the base may vary widely depending upon a variety of factors, such as the number of ingredients that are employed, the type of ingredients that are employed and the like, and may readily be determined by those of ordinary skill in the art.

One formula for a base cream (a cream emulsion base) that may be employed in the methods of the invention contains the components set forth below, and the concentrations thereof.

| Component | Weight Percent |
|---|---|
| Deionized water | 87.0-90.0% |
| Self-emulsifying wax N.F. (Fancor ® SEW-P)* | 7.75% |
| Methylparaben/propylparaben (preservative) | 0.25% |

*Available from The Fanning Corporation (Chicago, IL).

This base is prepared by separately heating the self-emulsifying wax and the water, containing preservatives, to about 80 degrees centigrade. Then, the oil phase (melted wax) is added slowly to the water phase with vigorous mixing. While mixing, the mixture is cooled. The cream begins to set up as the mixture cools. The mixture is continued cooling until the product reaches about 40 degrees centigrade. Mixing is then discontinued, and the product forms by standing at room temperature.

Water

The amount of water (deionized, saline, tap or other) that may be used in the compositions that are employed in the methods of the invention may vary widely depending upon the form of the compositions. Generally, for those forms of the compositions that include water, such as liquid cosmetic foundations, the amount of water that is employed is an amount that is effective for raising the total weight of the composition to 100 percent, and to provide the composition with a desired viscosity. This amount generally ranges from about 50 to about 90 weight percent of the total weight of the compositions, and preferably ranges from about 70 to about 80 weight percent, with about 75 weight percent being most preferred.

Optional Ingredients

The compositions that are employed in the methods of the invention may, in some cases, optionally, contain one or more other active or inactive ingredients, such as various vitamins, minerals, antioxidants, anti-inflammatory agents, antibacterial agents, antiviral agents, whitening agents, dispersing agents, thickening agents (xanthan gum, guar gum, gum arabic, methylcellulose, sodium carboxymethyl cellulose, carrageenan, starch and the like), anti-itch agents, polymers, emollients, pigments, cell activating agents, surfactants, emulsifiers, solvents (water, brine, polyethylene, glycerol, glycerine, mineral oil, other oils and the like), sunscreen agents, preservatives, perfume agents, colorants, collagen, glycerin, stearic acid, glycol stearate, lecithin, cetyl alcohol, talc, petrolatum jelly, cocoa butter, aloe vera, lanolin or other agents, for example, vitamin E (a-tocopherol, β-tocopherol, y-tocopherol, δ-tocopherol and the like). However, the compositions need not include any ingredients other than the Meadowestolide (active agent) and base (including one or more inactive ingredients).

Viscosity

The viscosity of the compositions that are employed in the methods of the invention, when in the form of a liquid or semi-liquid, may vary widely, depending upon the components that are present therein, the levels of components present therein, processing aids such as emulsifiers, and like considerations, but preferably ranges from about 5,000 cp to about 100,000 cp, and more preferably ranges from about 10,000 cp to about 60,000 cp. Viscosities of the compositions may be determined using a conventional viscometer.

PH

The pH of the compositions that are employed in the methods of the invention preferably ranges from about 2 to about 11, and more preferably ranges from about 5 to about 8, and may be adjusted using substances and methods known by those of skill in the art, such as acids (citric acid and the like) to decrease the pH or bases (sodium hydroxide and the like) to increase the pH.

Form of Compositions

The compositions that are employed in the methods of the invention may be present in any suitable or convenient state, including liquids, semi-solids and solids in, for example, a powder, creme, roll-on, aerosol, stick or other form. For example, the compositions may be in the form of a “leave-on” or “rinse-off” solution, lotion, cream, balsams, salve, oil, paste, ointment, gel, foam, solid, powder, antiperspirant, deodorant, aftershave, shaving lotion, or the like. In order to achieve maximum effectiveness, it is preferred that the compositions be in, and applied as, a “leave-on” form. However, if the compositions are in a “rinse-off” form, it is preferred that the compositions not be rinsed off (or otherwise removed from the skin) for a period of time that is sufficient for conditioning, which generally is at least about 4 minutes, and preferably is at least about 6 minutes, and more preferably is at least about 8 minutes, and still more preferably is at least about 10 minutes. Further, the compositions may be employed in a form of a skin-care product (skin cream, skin lotion, skin gel, skin foam, skin ointment, skin cleanser and the like), a cosmetic (base, foundation, concealer, loose powder, cake powder, rouge, etc.) or a veterinary product. Liquid forms of the compositions include, but are not limited to, solutions, dispersions, emulsions and microemulsions (water-in-oil, oil-in-water, oil-in-water-in-oil, water-in-oil-in-water, and the like).

Preparation of Compositions

The compositions that are employed in the methods of the invention can be prepared using customary methods and equipment known by those of skill in the art for preparing skin care products and cosmetics. For example, the Meadowestolide having a chemical structure set forth herein may be physically combined with a base to achieve the concentrations described herein by stirring together, or otherwise mixing or combining, the individual components. Preferably, sufficient agitation to achieve relative homogeneity is employed. Agitation may be achieved, for example, using a standard mixer, at a slow, moderate or even vigorous speed.

One formula for a composition that may be employed in the methods of the invention contains the components set forth below, and the concentrations thereof.

| Component | Weight Percent |
|---|---|
| Deionized water | 87.0-90.0% |
| Self-emulsifying wax N.F. (Fancor ® SEW-P) | 7.75% |
| Methylparaben/propylparaben (preservative) | 0.25% |
| Meadowestolide | 2.0% |

Other formulations for compositions that may be employed in the methods of the invention are provided in the Examples section set forth hereinbelow.

Packaging

The compositions that are employed in the methods of the invention may be packaged in any suitable manner for packaging skin care products or cosmetics, such as a plastic, metal or glass jar, bottle, tube, roll-on, compact case, cosmetic case or the like.

Quantity to be Administered and Application Rates

The compositions that are employed in the methods of the invention are preferably applied directly to the skin of a mammal in an amount, and for a number of applications, that are effective for enhancing (improving) the morphology, tone, texture and/or appearance of the mammal's skin.

The quantity of a composition to be administered to a mammal for each application depends upon the nature of the composition, the condition being treated and the area of the body involved, and may be determined by those of skill in the art using the information contained herein. The amount of a composition that will generally be effective for enhancing the morphology, tone, texture and/or appearance of the mammal's skin may vary widely, depending upon a variety of factors, such as the type, age, sex, genetic predisposition and general health of the mammal, as well as the mammal's sun exposure, and may readily be determined by those of skill in the art. The amount of a composition that is applied to the skin of a mammal per each application preferably ranges from about 1 to about 20 g or ml, and more preferably ranges from about 2 to about 12 g or ml, and still more preferably ranges from about 3 to about 8 g or ml, and even still more preferably ranges from about 4 to about 6 g or ml, with about 5 g or ml being most preferred. For example, about 5 g of the composition may be applied to the skin of a mammal from a suitable container or applicator and spread over, or rubbed into, the skin using the hands or fingers or a suitable application or other device.

The number of applications of a composition to the skin of a mammal that will generally be effective for producing an improvement in the morphology, tone, texture and/or appearance of the mammal's skin, and the period of time during which such applications are made, may vary widely, depending upon a variety of factors, such as the concentration of the Meadowestolide in the composition, the amount of the composition that is applied to the mammal's skin, the condition of the mammal's skin, the amount of the mammal's sun exposure and the type, age, sex, genetic predisposition and general health of the mammal, and may readily be determined by those of skill in the art. Typically, at least about three applications of the composition to the skin of the mammal (three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty and so forth applications) continuously over a period of at least about one day or week, or a series of days or weeks (one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty and so forth days or one, two, three, four, five, six, seven, eight, nine, ten, eleven, twelve, and so forth weeks) will be effective for improving the morphology, tone, texture and/or appearance of the mammal's skin. The compositions may, for example, be applied as a cream, lotion, ointment or other convenient form as frequently as once per hour and as infrequently as once per day.

Generally, the higher the number of applications of the composition to the skin of a mammal within a period of time, the greater an improvement will be observed or otherwise detected in the morphology, tone, texture and/or appearance of the skin, and the less time will be required for achieving such results. Although there generally is no limit to the number of applications of the composition that can be applied to the skin of a mammal, above a certain number of applications, no further improvement in the morphology, tone, texture and/or appearance of the skin may be observed or otherwise detected.

The table below shows examples of some of the application protocols that can be employed with the Meadowestolide employed in the present invention at concentrations that are described herein.

### EXAMPLES OF APPLICATION PROTOCOLS

| Number of | |
|---|---|
| Applications | Number of Days |
| 1 time each day | 3, 4, 5, 6, 7, 8, 9, 10, 11, 12, 13, 14, 15, 16, 17, 18, 19, 20, 21, 22, |
| | 23, 24, 25, 26, 27, 28, 29, 30, 31, 32, 33, 34, 35, 36 and so forth |
| 2 times each day | 1 and 1/2, 2, 3, 4, 5, 6, 7, 8, 9, 10, 11, 12, 13, 14, 15, 16, 17, 18, 19, |
| | 20, 21, 22, 23, 24, 25, 26, 27, 28, 29, 30, 31, 32, 33 and so forth |
| 3 times each day | 1, 2, 3, 4, 5, 6, 7, 8, 9, 10, 11, 12, 13, 14, 15, 16, 17, 18, 19, 20, 21, |
| | 22, 23, 24, 25, 26, 27, 28, 29, 30, 31, 32, 33, 34, 35 and so forth |
| 4 times each day | 1, 2, 3, 4, 5, 6, 7, 8, 9, 10, 11, 12, 13, 14, 15, 16, 17, 18, 19, 20, 21, |
| | 22, 23, 24, 25, 26, 27, 28, 29, 30, 31, 32, 33, 34, 35 and so forth |
| 5 times each day | 1, 2, 3, 4, 5, 6, 7, 8, 9, 10, 11, 12, 13, 14, 15, 16, 17, 18, 19, 20, 21, |
| | 22, 23, 24, 25, 26, 27, 28, 29, 30, 31, 32, 33, 34, 35 and so forth |
| 6 times each day | 1, 2, 3, 4, 5, 6, 7, 8, 9, 10, 11, 12, 13, 14, 15, 16, 17, 18, 19, 20, 21, |
| | 22, 23, 24, 25, 26, 27, 28, 29, 30, 31, 32, 33, 34, 35 and so forth |
| 7 times each day | 1, 2, 3, 4, 5, 6, 7, 8, 9, 10, 11, 12, 13, 14, 15, 16, 17, 18, 19, 20, 21, |
| | 22, 23, 24, 25, 26, 27, 28, 29, 30, 31, 32, 33, 34, 35 and so forth |
| 8 times each day | 1, 2, 3, 4, 5, 6, 7, 8, 9, 10, 11, 12, 13, 14, 15, 16, 17, 18, 19, 20, 21, |
| | 22, 23, 24, 25, 26, 27, 28, 29, 30, 31, 32, 33, 34, 35 and so forth |
| 9 times each day | 1, 2, 3, 4, 5, 6, 7, 8, 9, 10, 11, 12, 13, 14, 15, 16, 17, 18, 19, 20, 21, |
| | 22, 23, 24, 25, 26, 27, 28, 29, 30, 31, 32, 33, 34, 35 and so forth |
| 10 times each day | 1, 2, 3, 4, 5, 6, 7, 8, 9, 10, 11, 12, 13, 14, 15, 16, 17, 18, 19, 20, 21, |
| | 22, 23, 24, 25, 26, 27, 28, 29, 30, 31, 32, 33, 34, 35 and so forth |
| 11 times each day | 1, 2, 3, 4, 5, 6, 7, 8, 9, 10, 11, 12, 13, 14, 15, 16, 17, 18, 19, 20, 21, |
| | 22, 23, 24, 25, 26, 27, 28, 29, 30, 31, 32, 33, 34, 35 and so forth |
| 12 times each day | 1, 2, 3, 4, 5, 6, 7, 8, 9, 10, 11, 12, 13, 14, 15, 16, 17, 18, 19, 20, 21, |
| | 22, 23, 24, 25, 26, 27, 28, 29, 30, 31, 32, 33, 34, 35 and so forth |
| 13 times each day | 1, 2, 3, 4, 5, 6, 7, 8, 9, 10, 11, 12, 13, 14, 15, 16, 17, 18, 19, 20, 21, |
| | 22, 23, 24, 25, 26, 27, 28, 29, 30, 31, 32, 33, 34, 35 and so forth |
| 14 times each day | 1, 2, 3, 4, 5, 6, 7, 8, 9, 10, 11, 12, 13, 14, 15, 16, 17, 18, 19, 20, 21, |
| | 22, 23, 24, 25, 26, 27, 28, 29, 30, 31, 32, 33, 34, 35 and so forth |
| 15 times each day | 1, 2, 3, 4, 5, 6, 7, 8, 9, 10, 11, 12, 13, 14, 15, 16, 17, 18, 19, 20, 21, |
| | 22, 23, 24, 25, 26, 27, 28, 29, 30, 31, 32, 33, 34, 35 and so forth |
| 16 times each day | 1, 2, 3, 4, 5, 6, 7, 8, 9, 10, 11, 12, 13, 14, 15, 16, 17, 18, 19, 20, 21, |
| | 22, 23, 24, 25, 26, 27, 28, 29, 30, 31, 32, 33, 34, 35 and so forth |
| 17 times each day | 1, 2, 3, 4, 5, 6, 7, 8, 9, 10, 11, 12, 13, 14, 15, 16, 17, 18, 19, 20, 21, |
| | 22, 23, 24, 25, 26, 27, 28, 29, 30, 31, 32, 33, 34, 35 and so forth |
| 18 times each day | 1, 2, 3, 4, 5, 6, 7, 8, 9, 10, 11, 12, 13, 14, 15, 16, 17, 18, 19, 20, 21, |
| | 22, 23, 24, 25, 26, 27, 28, 29, 30, 31, 32, 33, 34, 35 and so forth |
| 19 times each day | 1, 2, 3, 4, 5, 6, 7, 8, 9, 10, 11, 12, 13, 14, 15, 16, 17, 18, 19, 20, 21, |
| | 22, 23, 24, 25, 26, 27, 28, 29, 30, 31, 32, 33, 34, 35 and so forth |
| 20 times each day | 1, 2, 3, 4, 5, 6, 7, 8, 9, 10, 11, 12, 13, 14, 15, 16, 17, 18, 19, 20, 21, |
| | 22, 23, 24, 25, 26, 27, 28, 29, 30, 31, 32, 33, 34, 35 and so forth |
| 21 times each day | 1, 2, 3, 4, 5, 6, 7, 8, 9, 10, 11, 12, 13, 14, 15, 16, 17, 18, 19, 20, 21, |
| | 22, 23, 24, 25, 26, 27, 28, 29, 30, 31, 32, 33, 34, 35 and so forth |
| 22 times each day | 1, 2, 3, 4, 5, 6, 7, 8, 9, 10, 11, 12, 13, 14, 15, 16, 17, 18, 19, 20, 21, |
| | 22, 23, 24, 25, 26, 27, 28, 29, 30, 31, 32, 33, 34, 35 and so forth |
| 23 times each day | 1, 2, 3, 4, 5, 6, 7, 8, 9, 10, 11, 12, 13, 14, 15, 16, 17, 18, 19, 20, 21, |
| | 22, 23, 24, 25, 26, 27, 28, 29, 30, 31, 32, 33, 34, 35 and so forth |
| 24 times each day | 1, 2, 3, 4, 5, 6, 7, 8, 9, 10, 11, 12, 13, 14, 15, 16, 17, 18, 19, 20, 21, |
| | 22, 23, 24, 25, 26, 27, 28, 29, 30, 31, 32, 33, 34, 35 and so forth |

It is preferable, but not necessary, that the various applications of the Meadowestolide that are applied each day be equally spaced with a 24-hour period. For example, it is preferable that two applications that are to be applied in one day are applied approximately 12 hours apart. It is preferable that three applications that are to be applied in one day are applied approximately 8 hours apart. It is preferable that four applications that are to be applied in one day are applied approximately 6 hours apart. It is preferable that five applications that are to be applied in one day are applied approximately 4.8 hours apart, and so forth. It is also preferable that days not be missed when a series of applications are to be made over a specified number of days.

As an example, after a composition containing about 4 weight percent of the Meadowestolide is applied one time per day to the skin of a mammal that requires, or could benefit from, skin treatment, an improvement in the morphology, tone, texture and/or appearance of the skin of the mammal will typically be observed or otherwise detected after a period of from about 4 to about 9 days. If the same composition is applied two times per day to the mammal's skin, an improvement in the morphology, tone, texture and/or appearance of the skin of the mammal will typically be observed or otherwise detected after a period of from about 4 to about 8 days. If the same composition is applied three times per day to the mammal's skin, an improvement in the morphology, tone, texture and/or appearance of the skin of the mammal will typically be observed or otherwise detected after a period of from about 4 to about 7 days. If the same composition is applied four times per day to the mammal's skin, an improvement in the morphology, tone, texture and/or appearance of the skin of the mammal will typically be observed or otherwise detected after a period of from about 4 to about 6 days. If the same composition is applied five times per day to the mammal's skin, an improvement in the morphology, tone, texture and/or appearance of the skin of the mammal will typically be observed or otherwise detected after a period of from about 4 to about 5 days.

As another example, if a composition containing about 6 weight percent of the Meadowestolide is applied one time per day to the skin of the mammal, an improvement in the morphology, tone, texture and/or appearance of the skin of the mammal will typically be observed or otherwise detected after a period of from about 3 to about 8 days. If the same composition is applied two times per day to the mammal's skin, an improvement in the morphology, tone, texture and/or appearance of the skin of the mammal will typically be observed or otherwise detected after a period of from about 3 to about 7 days. If the same composition is applied three times per day to the mammal's skin, an improvement in the morphology, tone, texture and/or appearance of the skin of the mammal will typically be observed or otherwise detected after a period of from about 3 to about 6 days. If the same composition is applied four times per day to the mammal's skin, an improvement in the morphology, tone, texture and/or appearance of the skin of the mammal will typically be observed or otherwise detected after a period of from about 3 to about 5 days. If the same composition is applied five times per day to the mammal's skin, an improvement in the morphology, tone, texture and/or appearance of the skin of the mammal will typically be observed or otherwise detected after a period of from about 3 to about 4 days.

As yet another example, if a composition containing about 9 weight percent of the Meadowestolide is applied one time per day to the skin of the mammal, an improvement in the morphology, tone, texture and/or appearance of the skin of the mammal will typically be observed or otherwise detected after a period of from about 2 to about 7 days. If the same composition is applied two times per day to the mammal's skin, an improvement in the morphology, tone, texture and/or appearance of the skin of the mammal will typically be observed or otherwise detected after a period of from about 2 to about 6 days. If the same composition is applied three times per day to the mammal's skin, an improvement in the morphology, tone, texture and/or appearance of the skin of the mammal will typically be observed or otherwise detected after a period of from about 2 to about 5 days. If the same composition is applied four times per day to the mammal's skin, an improvement in the morphology, tone, texture and/or appearance of the skin of the mammal will typically be observed or otherwise detected after a period of from about 2 to about 4 days. If the same composition is applied five times per day to the mammal's skin, an improvement in the morphology, tone, texture and/or appearance of the skin of the mammal will typically be observed or otherwise detected after a period of from about 2 to about 3 days.

As another example, if a composition containing about 12 weight percent of the Meadowestolide is applied one time per day to the skin of the mammal, an improvement in the morphology, tone, texture and/or appearance of the skin of the mammal will typically be observed or otherwise detected after a period of from about 1 to about 6 days. If the same composition is applied two times per day to the mammal's skin, an improvement in the morphology, tone, texture and/or appearance of the skin of the mammal will typically be observed or otherwise detected after a period of from about 1 to about 5 days. If the same composition is applied three times per day to the mammal's skin, an improvement in the morphology, tone, texture and/or appearance of the skin of the mammal will typically be observed or otherwise detected after a period of from about 1 to about 4 days. If the same composition is applied four times per day to the mammal's skin, an improvement in the morphology, tone, texture and/or appearance of the skin of the mammal will typically be observed or otherwise detected after a period of from about 1 to about 3 days. If the same composition is applied five times per day to the mammal's skin, an improvement in the morphology, tone, texture and/or appearance of the skin of the mammal will typically be observed or otherwise detected after a period of from about 1 to about 2 days.

As still another example, if a composition containing about 15 weight percent of the Meadowestolide is applied one time per day to the skin of the mammal, an improvement in the morphology, tone, texture and/or appearance of the skin of the mammal will typically be observed or otherwise detected after a period of from about 0.5 to about 5 days. If the same composition is applied two times per day to the mammal's skin, an improvement in the morphology, tone, texture and/or appearance of the skin of the mammal will typically be observed or otherwise detected after a period of from about 0.5 to about 4 days. If the same composition is applied three times per day to the mammal's skin, an improvement in the morphology, tone, texture and/or appearance of the skin of the mammal will typically be observed or otherwise detected after a period of from about 0.5 to about 3 days. If the same composition is applied four times per day to the mammal's skin, an improvement in the morphology, tone, texture and/or appearance of the skin of the mammal will typically be observed or otherwise detected after a period of from about 0.5 to about 2 days. If the same composition is applied five times per day to the mammal's skin, an improvement in the morphology, tone, texture and/or appearance of the skin of the mammal will typically be observed or otherwise detected after a period of from about 0.5 to about 1 day.

It is preferable that at least 15 applications of the composition are applied to the skin of a mammal over a period of about 5 days, for example, 3 applications of the composition to the skin per day for a period of 5 days. It is more preferable that at least 60 applications of the composition are applied to the skin of a mammal over a period of about 15 days, for example, 4 applications of the composition to the skin per day for a period of 15 days. It is still more preferable that at least 120 applications of the composition are applied to the skin of a mammal over a period of about 30 days, for example, 4 applications of the composition to the skin per day for a period of 30 days.

Sources of Ingredients

All of the ingredients, materials and equipment employed in the examples, and generally employed in the methods of the invention, are commercially available from sources known by those of skill in the art, such as The Fanning Corporation (Chicago, Ill.), Botagenics, Inc. (Northridge, Calif.), Base Formula, Ltd. (Melton Mowbray, England), Well Naturally Products, Ltd. (Blaine, Wash.), Essential Wholesale (Clackamas, Oreg.), Urist Cosmetics, Inc. (Richmond, Calif.), Sciencelab.com, Inc. (Houston, Tex.), Fluka Chemical and Biochemical Co. (Ronkonkoma, N.Y.), Sony North America (New York, N.Y.), NOVA Technology Corporation (Portsmouth, N.H.) and ServoMed (Sweden).

The following examples describe and illustrate the methods of the present invention. These examples are intended to be merely illustrative of the present invention, and not limiting thereof in either scope of spirit. Those of skill in the art will readily understand that variations of certain of the conditions and/or steps employed in the procedures described in the examples can be employed.

### Example 1

### Preparation of Meadowestolide

The Meadowestolide that is employed in the methods of the invention, the structure of which is set forth hereinbelow, was synthesized from the meadowfoam seed oil fatty acids that are set forth below, which are naturally present in meadowfoam seed oil, and scaled-up using an acid catalyzed condensation reaction process (to form a dimeric ester) followed by a series of high vacuum distillations (to purify the material). Unsaturation at the Δ⁵⁻⁶ and Δ^{13,14} locations enables the synthesis of the Meadowestolide.

| | Weight Percent of Total Fatty |
|---|---|
| Meadowfoam Seed Oil Fatty Acids | Acids Present in Meadowfoam Seed Oil |
| CH₃—(CH₂)₁₃—(CH)₂—(CH₂)₃—COOH | 63% |
| Δ⁵ 20:1 | |
| CH₃—(CH₂)₁₅—(CH)₂—(CH₂)₃—COOH | 4% |
| Δ⁵ 22:1 | |
| CH₃—(CH₂)₇—(CH)₂—(CH₂)₁₁—COOH | 12% |
| Δ¹³ 22:1 | |
| CH₃—(CH₂)₇—(CH)₂—(CH₂)₆—(CH)₂—(CH₂)₃—COOH | 17% |
| Δ^{5,13} 22:1 | |

Meadowestolide

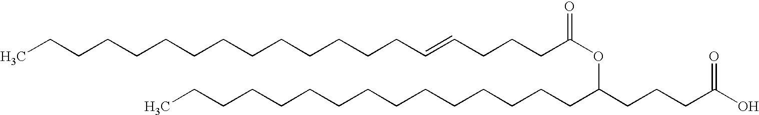

Analytical results indicated a high level of purity and extended stability for the Meadowestolide. The purified Meadowestolide was identified and subsequently quantified by infrared spectroscopy and HPLC coupled with an evaporative light-scattering detector.

This Meadowestolide is commercially available from The Fanning Corporation (Chicago, Ill.).

### Example 2

### Preparation of Skin Cream Containing Meadowestolide

A composition including the Meadowestolide prepared in Example 1 was prepared using a skin cream base. The composition contained the components set forth below, and the concentrations thereof.

| Component | Weight Percent |
|---|---|
| Deionized water | 87.0-90.0% |
| Self-emulsifying wax N.F. (Fancor ® SEW-P) | 7.75% |
| Methylparaben/propylparaben (preservative) | 0.25% |
| Meadowestolide | 2.0% |

The composition was prepared by separately heating to 80 degrees centigrade: (a) the Self-emulsifying wax N.F. and the Meadowestolide; and (b) the water containing the preservatives. Then, the oil phase (melted wax) was slowly added to the water phase with vigorous mixing, with a cooling of the mixture commencing while mixing. A cream began to set up as the mixture cooled. The mixture was cooled until it reached about 40 degrees centigrade. Mixing was discontinued and the final skin cream product was formed by standing at room temperature.

### Example 3

### In Vitro Water Binding Experiments

It was postulated that, as a result of the structure of the Meadowestolide prepared in Example 1, which has a carboxylic acid function and an ester linkage in juxtaposition to one another, a considerable degree of hydrogen-bonding and consequent water binding should be evident. Consequently, in vitro experiments to test this theory employing the Meadowestolide, as well as the carefully selected comparative control compounds described below, were conducted.

### Control Compounds

(a) the triglyceride oil (meadowfoam seed oil) from which the Meadowestolide was derived, which has the following chemical structure: 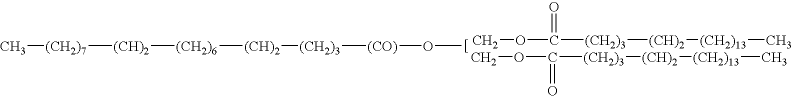
(b) a fatty acid from which the Meadowestolide was formed (Meadowfoam fatty acid, CH₃—(CH₂)₁₅—(CH)₂—(CH₂)₃—COOH); and
(c) Joj oba oil (CH₃—(CH₂)₇—(CH)₂—(CH₂)₁₀—O—(CO)—(CH₂)₁₀—(CH)₂—(CH₂)₇—CH₃), which has no carboxylic acid groups.

The Meadowestolide prepared in Example 1 (3 ml) and the control substances (3 ml) were carefully layered over distilled water (10 ml) using a long-nosed pipette, hand mixed for 30 seconds and photographed using a Sony Digital DSC-F505V Camera equipped with a Carl Zeiss Vario-Sonner lens. The degree of water retention was then examined by measuring the “loss on drying” (water removed from lipid phase at 100° C.). The upper lipid phase was completely removed using a long-nose pipette and analyzed for water content by “loss on drying” using a drying oven at 105 degrees centigrade until there was no longer any change in weight.

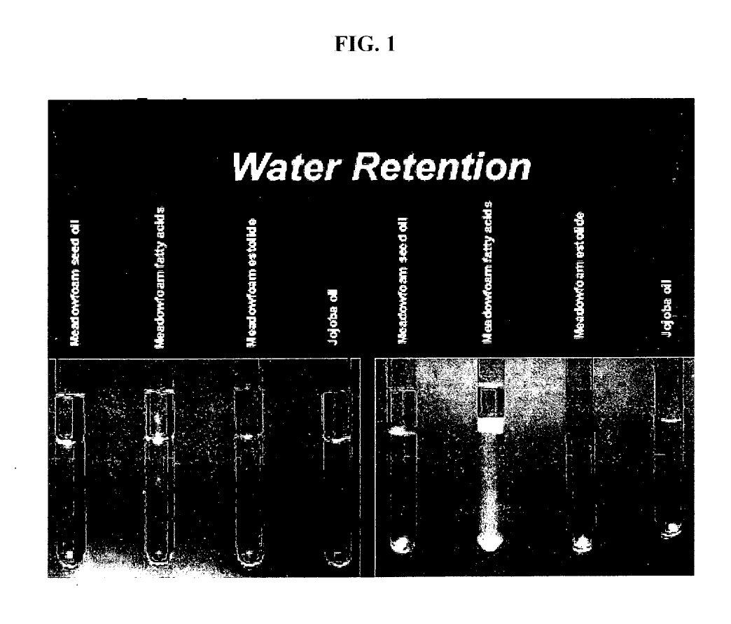

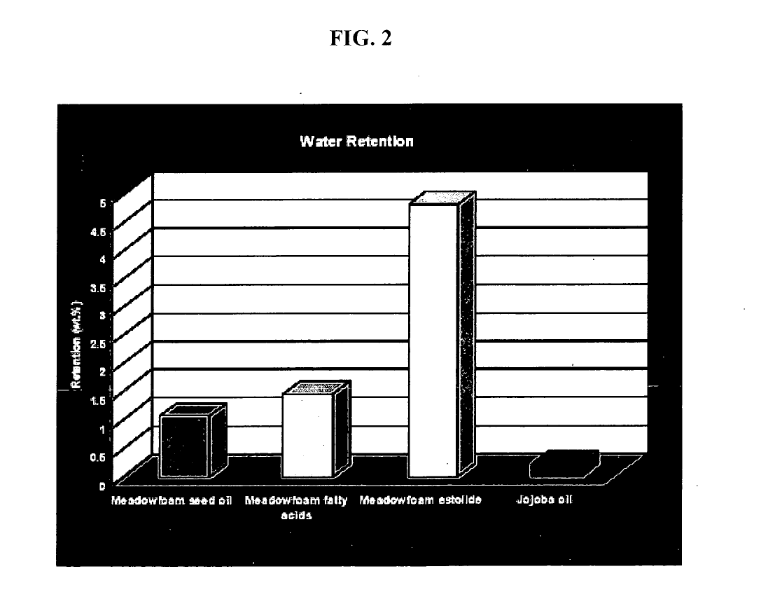

During these in vitro experiments, it was observed that a measurable amount of water can partition into the Meadowestolide and bind to its polar “hydrophilic center” (—CO—O—CH—(CH₂)₃—CO—OH portion). The water retention results of these experiments are shown in FIG. 1 (results of water retention of Meadowfoam seed oil, Meadowfoam fatty acid, Meadowestolide and Jojoba oil in the form of a photograph) and in FIG. 2 (results of water retention of Meadowfoam seed oil, Meadowfoam fatty acid, Meadowestolide and Jojoba oil in the form of a graph). The left side of FIG. 1 shows the layers prior to mixing, and the right side of FIG. 1 shows the layers after mixing. FIG. 1 and FIG. 2 show that, although the Meadowfoam seed oil and the meadowfoam fatty acid each retained a small quantity of water, only the Meadowestolide was capable of binding water to a significant extent. Surprisingly and unexpectedly, the Meadowestolide was found to be capable of binding approximately four times more water than the Meadowfoam seed oil and the meadowfoam fatty acid, and approximately nine times more water than the Jojoba oil.

### Example 4

### In Vivo Transepidermal Water Loss and Tissue Hydration Experiments

The mammalian skin is an anatomical and physiological barrier between an animal and its environment. Although the skin combines a number of barrier properties to perform this role, critical among these barrier properties is an ability to limit the rate of water loss from the skin. Maintenance of homeostatic levels of water is imperative for many physiological processes in the body, and the skin, in particular, is acutely sensitive to hydration levels. Thus, the skin and, in particular, the epidermis, has evolved a number of characteristics that, in combination, serve to limit a concession of water to the outside world. The stratified structure of the outer cell layers, and the rich extracellular lipid, in this region provide a boundary to all but a low level of insensate aqueous transfer. Nonetheless, as a result of this basal level of water loss, a paracutaneous zone is maintained around the entire body, which is known as a water vapor boundary layer. In the case of animals that have a hair coat, this creates a region of elevated humidity within the coat.

Transepidermal water loss (TEWL) describes the total amount of water lost through the skin, which is a loss that occurs constantly by passive diffusion through the epidermis. Although TEWL is a normal physiological phenomenon, if it rises too high, the skin can become dehydrated, disrupting its form and function and, potentially, leading to infection or transepidermal passage of deleterious agents.

During the in vitro experiments that are described in Example 3, it was observed that a measurable amount of water can partition into the Meadowestolide described therein and bind to its polar “hydrophilic center.” Therefore, similar studies to those described in Example 3 were conducted in vivo using appropriate comparative control substances.

TEWL and tissue hydration (of the stratum comeum) experiments were designed to evaluate the moisture retention properties of the Meadowestolide described in Example 1 in comparison with a relevant oil and a non-polymerized ester. Thus, for these studies, the natural oil to select as a control was Meadowfoam seed oil since it was the precursor oil from which the Meadowestolide was derived. The ester selected for comparison was the guerbet ester of meadowfoam fatty acid (“Meadowester GME”) because it has approximately the same molecular weight (size) as the Meadowestolide, but does not contain a carboxylic acid/ester binding site, and, as a result of its branched chain, it is quite fluid at room temperature. Thus, in these experiments, the following substances were compared: (a) Meadowfoam seed oil (triglyceride oil); (b) a long-chain guerbet ester of meadowfoam fatty acid (CH₃(CH₂)₉CH((CH)₂O(CO)(CH₂)₂₂CH₃)((CH₂)₇CH₃); and (c) the Meadowestolide prepared in Example 1.

These experiments were conducted to assess and compare the moisturizing efficacy of the Meadowestolide and appropriate controls on human skin using a NOVA Technology Corporation (Portsmouth, N.H.) Dermal Phase Meter 9003 (DPM) and a ServoMed Evaporimeter (ServoMed, Sweden). The combined use of these instruments assesses the moisturizing and barrier enhancement properties of topically applied materials. Typically, good moisturizers will exhibit the classical inverse relationship with an increased tissue hydration, as recorded by electrical capacitance and decreased TEWL. Twenty subjects completed these TEWL and tissue hydration experiments.

Twenty panel subjects (10 males and 10 females) ranging in age from 30 to 45 years were employed in these experiments. One application of a test substance was applied as neat (100%) to the forearm and was left on the forearm for approximately 2 minutes.

A ServoMed Evaporimeter was used to measure transepidermal water loss (TEWL). A hand-held probe on this meter samples the relative humidity at two points above the skin surface, thereby allowing the rate of water loss to be calculated from the humidity gradient. Measurements are recorded as g/m2/hr. Each TEWL measurement was taken after 45 seconds of site stabilization. The instrument operator wore a surgical mask over the nose and mouth to minimize the effects of breathing and a padded, well-insulated glove to minimize the influence of body temperature on the probe.

In the tissue hydration experiments, a Dermal Phase Meter 9003 was used to quantify moisture content in the stratum comeum (SC) by an electrical capacitance method. The measurement has no units, but is proportional to the dielectric constant of the surface layers of the skin, and increases as the skin becomes more hydrated. The Dermal Phase Meter 9003 numbers are directly related to the electrical capacitance of the skin measured as picoFarads (pF).

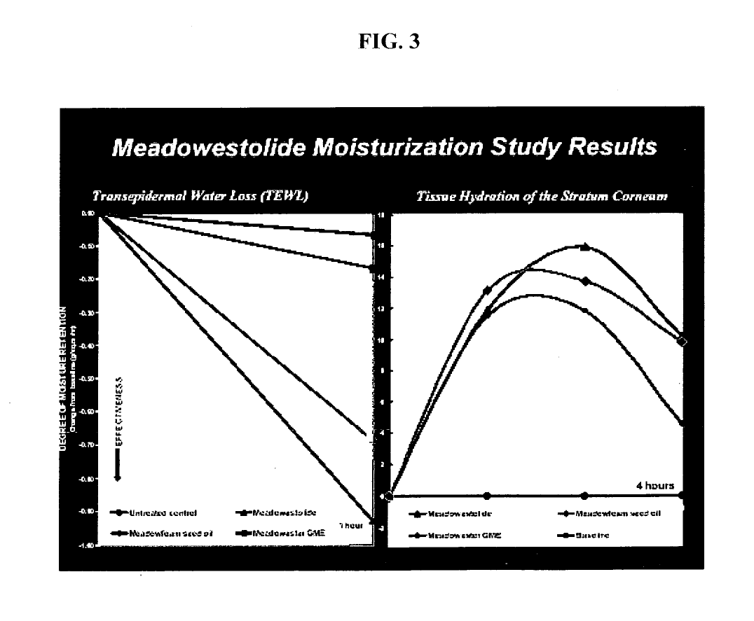

The results of the TEWL and tissue hydration experiments are present in FIG. 3, and clearly show that the Meadowestolide is very efficient in preventing water loss, and retaining water, in epidermal tissue. The decrease in TEWL, and the improved hydration, of the Meadowestolide in comparison with Meadowfoam seed oil, indicate that there is more than simply occlusivity accounting for these results. While not wishing to be bound by any theory, it is likely that the “hydrophilic center” of the Meadowestolide is actively holding onto water through hydrogen bonding. Because the Meadowestolide structure is oriented in a manner that concentrates polarity at one end of the molecule and, therefore, presents multiple sites for H-bonding, its hydration capability exceeds that of a simple ester, even when the molecular sizes are reasonably equivalent.

### Example 5

### Testing of Skin with Skin Cream Containing Meadowestolide

In these topical application experiments, a simple cream base containing no conditioning or active agents other than the test material (Meadowestolide skin cream prepared in Example 2, but containing varying quantities of Meadowestolide), and appropriate control skin creams (skin cream base alone or skin cream base plus water, glycerin or petrolatum), were applied to the skin of twenty human beings in varying quantities and at varying application rates.

Subiects and Application Protocols

The panel of subjects used for these studies were both male and female ranging from twenty to sixty-three years of age. Initial applications of the Meadowestolide skin cream and control skin creams, and instructions for subsequent applications, were supervised by a professional aesthetician. A variety of application procedures were employed with respect to number of applications of a test material and duration of applications in an attempt to reflect actual market usage of these types of products, as well as to determine the extent to which observed results were a function of duration and/or frequency of use. The application protocols (frequency and duration of use), and concentration of test material used, varied as is discussed below. Each member of the panel applied Meadowestolide skin cream, or Meadowlactone skin cream plus an appropriate control, to their face, hands or forearms.

Although application studies were performed on approximately 20 subjects, only 2 of the subjects are discussed herein because the results for these two subjects were typical of the results obtained for all of the subjects.

Image Capture and Processing Instrumentation

The following image capture and processing instrumentation were employed in these experiments:
(1) a Sony Cyber-Shot digital still camera equipped with an X1.4 Teleconversion lens (VCL-1452H) (Sony North America, New York, N.Y.); and
(2) a “Micro-Topological Epidermal Imaging” (MTEI) instrument using a portable high magnification fiber optic microscope (to achieve a higher magnification in vivo visualization of skin tissue).
   External lighting sources were employed to facilitate the visualization of epidermal surface topography. In some instances, optical shadow casting techniques were used to better define surface topology.

Experiments and Results

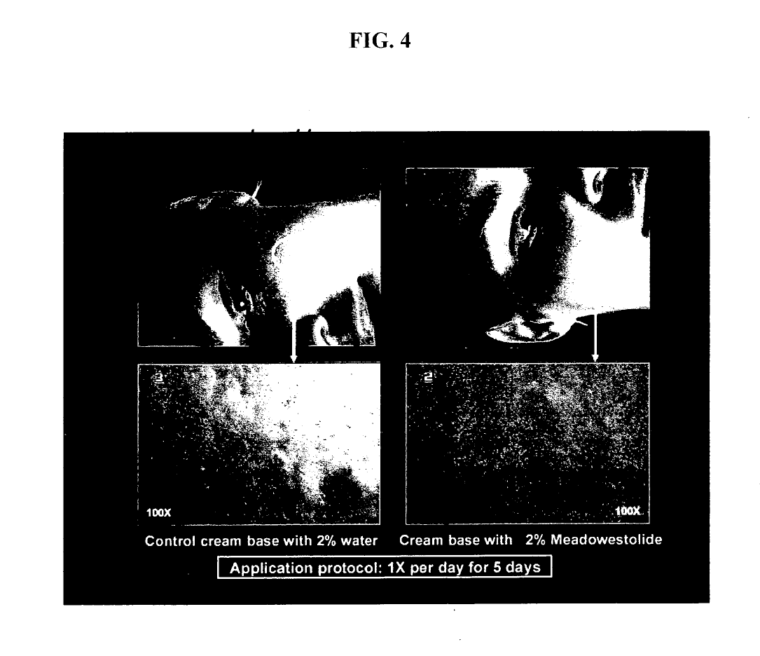

In a first experiment, a female subject applied the Meadowestolide skin cream containing 2% of Meadowestolide (active ingredient) and a control skin cream (the same skin cream formulated with 2% water in place of the Meadowestolide) to her face once per day for a period of 5 days. The right side of her face received the active product while the left side of her face was treated with the control product. The results of this experiment are shown in FIG. 4 (photographs of the right and left sides of the subject's face both without magnification and with 1OOx magnification). FIG. 4 clearly shows that the right side of the subject's face (treated with Meadowestolide skin cream, and shown in the upper and lower right photographs) has a significant improvement in the surface topology of the skin. The aesthetician observed a marked improvement in the condition of the epidermal tissue in the right side of the face, noting that the right side was smoother and had better tone than did the left side (shown in the upper and lower left photographs). Further, the improved condition, tone and appearance of the skin were clearly obvious to the subject.

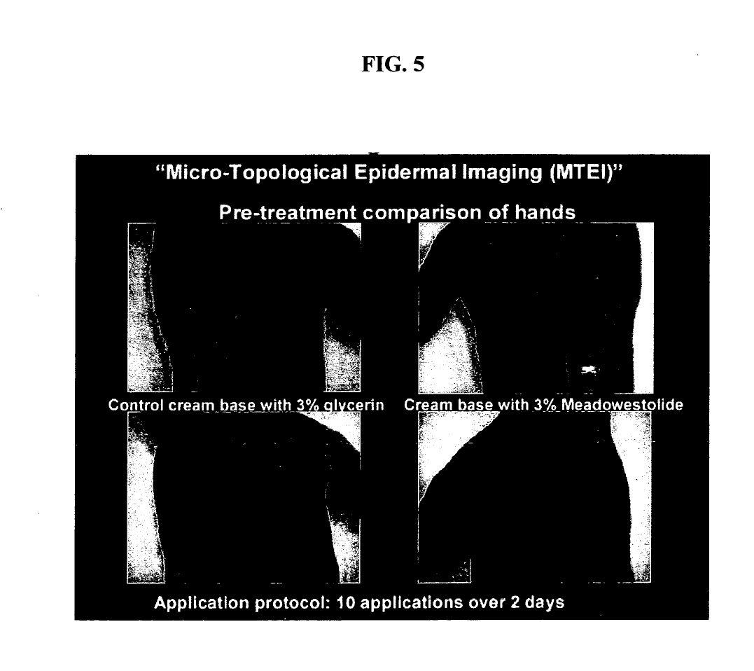

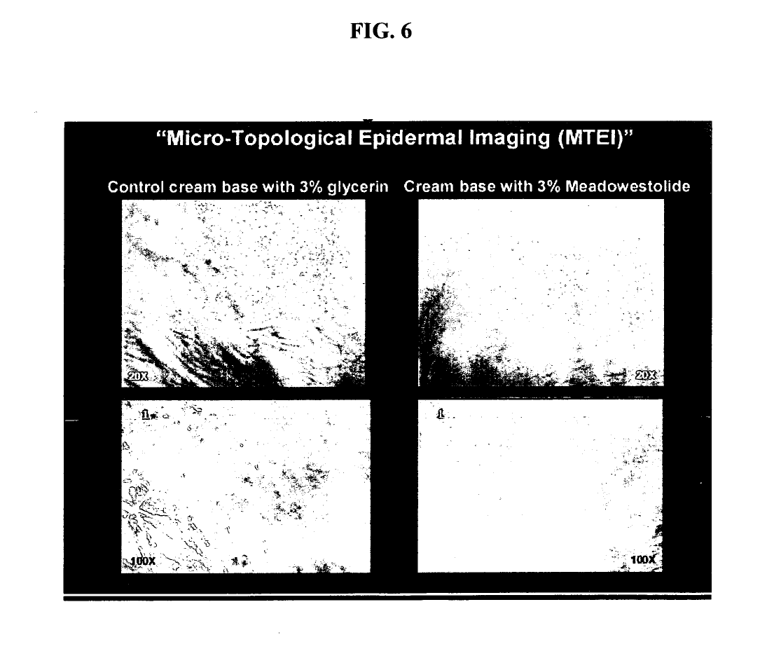

An even greater challenge is presented when attempting to moisturize the skin of a geriatric subject. In a second experiment, the Meadowestolide skin cream containing 3% of Meadowestolide (active ingredient) and a control (the same skin cream base formulated with 3% glycerin in place of the Meadowestolide) were separately applied to different hands of a 63-year-old man five times per day (approximately every 3 hours) over a period of two days (i.e., ten applications over a two-day period). The Micro-Topological Epidermal Imaging (MTEI) results of this experiment are shown in FIG. 5 and FIG. 6 as photographs.

In FIG. 5, the upper two photographs show both of the subject's hands prior to any application of any product. The lower right photograph shows the subject's left hand after ten applications of a Meadowestolide skin cream base containing 3 weight percent Meadowestolide thereto (over a period of two days). The lower left photograph shows the subject's right hand after ten applications of a control skin cream base containing 3 weight percent glycerine in place of the Meadowestolide (over a period of two days).

FIG. 6 shows the same hands as those shown in the lower two photographs of FIG. 5. In FIG. 6, the upper right photograph shows the subject's left hand at a magnification of 20× after the ten applications of the Meadowestolide skin cream (containing 3 weight percent Meadowestolide, and applied five times per day over a period of two days). The lower right photograph shows the same hand as in the upper right photograph, with the exception that the magnification is 100×. The upper left photograph shows the subject's right hand at a magnification of 20× after ten applications of the control skin cream (the same skin cream base formulated with 3% glycerin in place of the Meadowestolide, and applied five times per day over a period of two days). The lower left photograph shows the same hand as in the upper left photograph, with the exception that the magnification is 100×.

FIG. 5 and FIG. 6 clearly show that, although the control skin cream (containing 3% glycerin in a simple skin cream base) appeared to have improved the overall appearance and texture of the subject's right hand, a significantly greater degree of improvement, in terms of moisturization, toning and elasticity, was achieved with the Meadowestolide skin cream (containing 3 weight percent of Meadowestolide in the same skin cream base) that was applied to the subject's left hand.

In the above-described experiments, in every case, a noticeable improvement in skin feel and texture for the area treated with the Meadowestolide skin cream product was observed and/or detected in comparison with the area treated with the control skin cream product (treated with the base cream alone or with base cream plus water, glycerin or petrolatum).

### Example 6

Muscle Relaxant Skin Formulation

| | Ingredient | Weight % | INCI Name | Function |
|---|---|---|---|---|
| 1. | Deionized Water | q.s. | Water | Diluent |
| 2. | Methylparaben, | 0.25 | Methylparaben, | Preservative |
| | Propylparaben | | Propylparaben | |
| 3. | Fanwax ™ Sew-P | 5.50 | Emulsifying wax NF | Emulsifier/ |
| | | | | Thickener |
| 4. | Fancor ® Lanolin | 5.55 | Lanolin | Emollient/ |
| | USP | | | Conditioner |
| 5. | White Petrolatum | 3.50 | Petrolatum | Conditioner/ |
| | | | | Protectant |
| 6. | Mineral Oil | 2.00 | Mineral Oil | Emollient/ |
| | | | | Lubrication |
| 7. | Meadowestolide | 2.00 | Meadowfoam Estolide | Active Agent |
| 8. | Menthol | 0.55 | Menthol | Cooling |
| | | | | Agent/ |
| | | | | Medicinal |
| 9. | Eucalyptus Oil | 0.50 | Eucalyptus | Cooling |
| | | | | Agent/ |
| | | | | Medicinal |
| 10. | Camphor | 1.00 | Camphor | Cooling |
| | | | | Agent/ |
| | | | | Medicinal |
| 11. | Peppermint Oil | 1.00 | Peppermint | Calming |
| | | | | Agent/ |
| | | | | Medicinal |
| 12. | Phenoxyethol | 0.40 | Phenoxyethanol | Preservative |
| 13. | Symphytum | 0.25 | Symphytum Officinale | Herbal |
| | Officinale | | | |

| Extract | Extract/Botanicals |
|---|---|
| Plantago Ovata Seed Extract | Plantago Ovata Seed Extract |
| Sambucus Nigra Flower Extract | Sambucus Nigra Flower Extract |
| Equisetum Arvense Extract | Equisetum Arvense Extract |
| Calendula Officinalis Flower Extract | Calendula Officinalis Flower |
| | Extract |
| Salvia Officinalis (Sage) Leaf Extract | Salvia Officinalis (Sage) Leaf |
| | Extract |
| Geramium Maclatum Extract | Geramium Maclatum Extract |
| Panax Ginseng Root Extract | Panax Ginseng Root Extract |
| Honey Extract | Honey Extract |

Preparative Procedure:
Ingredients 1. and 2. are heated to 75° C. in a main vessel. Separately, ingredients 3., 4., 5., 6. and 7. are heated to 75° C., added to the water phase and mixed strongly for 15 minutes. The mixture is then slowly agitated and cooled to 55° C. Ingredients 8., 9., 10. and 11 are added, with mixing continued until homogenous. The mixture is cooled to 30° C. Ingredients 12. and 13 are added, and cooling is continued until the mixture reaches room temperature.
Ingredients 3., 4. and 7. are commercially available from The Fanning Corporation (Chicago, Ill.).
   Characteristics:
pH: 4.00-5.50
Viscosity: 12,000-16,000 cps @ 25° C.
Appearance: White to off white thick cream.

### Example 7

Hand and Body Moisturizing Lotion

| | Ingredient | Weight % | INCI Name | Function |
|---|---|---|---|---|
| 1. | Deionized Water | 71.10 | Water | Diluent |
| 2. | Glycerin 99% | 3.00 | Glycerin | Humectant |
| 3. | Mineral Oil | 8.00 | Mineral Oil | Emollient |
| 4. | Fanwax ® SEW-P | 3.00 | Emulsifying | Emulsifier |
| | | | Wax NF | |
| 5. | Cetearyl Alcohol | 1.50 | Cetearyl Alcohol | Thickener |
| 6. | Meadowestolide | 2.00 | Meadowfoam | Active Agent |
| | | | Estolide | |
| 7. | Phenoxyethanol, | 1.00 | Phenoxyethanol, | Preservative |
| | Methylparaben, | | Methylparaben, | |
| | Propylparaben, | | Propylparaben, | |
| | Butylparaben, and | | Butylparaben, and | |
| | Ethylparaben | | Ethylparaben | |
| 8. | Fancorsil ® HA | 10.00 | Glyceryl | Emulsifier/ |
| | Super | | Polyacrylate | Thickener |
| | | | Dimethicone, | |
| | | | Cyclomethicone | |
| 9. | DMDM Hydantoin | 0.40 | DMDM Hydantoin | Preservative |

Preparative Procedure:
Ingredients 1. and 2. were heated to 75° C. in a main vessel. Separately, ingredients 3., 4., 5. and 6. were heated to 80° C. and added to the water phase, and mixed well for 15 minutes. Using slow agitation, the mixture was cooled to 50° C. Ingredients 7. and 8. were added and mixed until homogenous. Ingredient 9. was added and cooling was continued until the mixture reached 30° C.
Ingredients 4., 6. and 8. are commercially available from The Fanning Corporation (Chicago, Ill.).
   Characteristics:
pH: 6-7;
Viscosity: 3,000-5,000 cps.

### Example 8

Hand Moisturizing Cream

| | Ingredient | Weight % | INCI Name | Function |
|---|---|---|---|---|
| 1. | Deionized Water | 70.10 | Water | Diluent |
| 2. | Glycerin 99% | 3.00 | Glycerin | Humectant |
| 3. | Mineral Oil | 5.00 | Mineral Oil | Emollient |
| 4. | Fanwax ® Sew-P | 3.00 | Emulsifying Wax, | Emulsifier |
| | | | NF | |
| 5. | Fancol ® VB | 5.00 | Limnanthes Alba | Conditioner |
| | | | (Meadowfoam) | |
| | | | Seed oil, | |
| | | | Butyrospermum | |
| | | | Parkii | |
| | | | (Shea Butter) | |
| | | | Extract | |
| 6. | Cetearyl Alcohol | 1.50 | Cetearyl Alcohol | Thickener |
| 7. | Meadowestolide | 3.00 | Meadowfoam | Active Agent |
| | | | Estolide | |
| 8. | Phenoxyethanol, | 1.00 | Phenoxyethanol, | Preservative |
| | Methylparaben, | | Methylparaben, | |
| | Ethylparaben, | | Ethylparaben, | |
| | Butylparaben, and | | Butylparaben, and | |
| | Propylparaben | | Propylparaben | |
| 9. | Fancorsil ® HA | 8.00 | Glyceryl | Emulsifier/ |
| | Super | | Polyacrylate | Thickener |
| 10. | DMDM Hydantoin | 0.40 | DMDM Hydantoin | Preservative |

Preparative Procedure:
Ingredients 1. and 2. are heated to 75° C. in a main vessel. Separately, ingredients 3., 4., 5. and 6 are heated to 80° C. and added to the water phase, and then mixed well for 15 minutes. Using slow agitation, the mixture is cooled to 50° C. Ingredients 7. and 8. are added and mixed until homogenous. Ingredients 9. and 10. are added and mixed, with cooling continued to 30° C.
Ingredients 4., 5., 7. and 9. are commercially available from The Fanning Corporation (Chicago, Ill.).
   Characteristics:
pH: 6-7
Viscosity: 5,000-8,000 cps

### Example 9

Hand Moisturizing Cream

| | Ingredient | Weight % | INCI Name | Function |
|---|---|---|---|---|
| 1. | Deionized Water | 75.35 | Water | Diluent |
| 2. | Glycerin 99% | 3.00 | Glycerin | Humectant |
| 3. | Mineral Oil | 5.00 | Mineral Oil | Emollient |
| 4. | Fancor ® Uni- | 3.00 | Cetearyl Alcohol, | Emulsifier |
| | Embase | | Dimethicone PEG-8 | |
| | | | Meadowfoamate, | |
| | | | Meadowfoam | |
| | | | Amidopropyldimethyl | |
| | | | Betaine | |
| 5. | Fancol ® VB | 5.00 | Meadowfoam | Conditioner |
| | | | (Limnanthes | |
| | | | Alba) Seed oil, Shea | |
| | | | Butter | |
| | | | (Butyrospermum | |
| | | | Parkii) Extract | |
| 6. | Cetearyl Alcohol | 1.50 | Cetearyl Alcohol | Thickener |
| 7. | Meadowestolide | 2.00 | Meadowfoam | Active Agent |
| | | | Estolide | |
| 8. | Phenoxyethanol, | 0.75 | Phenoxyethanol, | Preservative |
| | Methylparaben, | | Methylparaben, | |
| | Butylparaben, | | Butylparaben, | |
| | Ethylparaben, and | | Ethylparaben, and | |
| | Propylparaben | | Propylparaben | |
| 9. | Fancorsil ® HA | 4.00 | Glyceryl Polyacrylate | Emulsifier/ |
| | Super | | | Thickener |
| 10. | DMDM | 0.40 | DMDM Hydantoin | Preservative |
| | Hydantoin | | | |

Preparative Procedure:
Ingredients 1. and 2. are heated to 75° C. in a main vessel. Separately, ingredients 3., 4., 5. and 6. are heated to 80° C. and added to the water phase. The mixture is mixed well for 15 minutes. Using slow agitation, the mixture is cooled to 50° C. Ingredients 7. and 8. are added to, the mixture and mixed until homogenous. Ingredients 9. and 10. are added to the mixture, and the mixture is cooled to 30° C.
Ingredients 4., 5., 7. and 9. are commercially available from The Fanning Corporation (Chicago, Ill.).
   Characteristics:
pH: 4.0-5.0
Viscosity: 10,000-18,000 cps

### Example 10

Hand/Skin Cream Moisturizing Conditioner

| Ingredient | | Weight % | INCI Name | Function |
|---|---|---|---|---|
| 1. | Deionized Water | 85.35 | Water | Diluent |
| 2. | Glycerin 99% | 3.00 | Glycerin | Humectant |
| 3. | Methylparaben, | 0.25 | Methylparaben, | Preservative |
| | Propylparaben | | Propylparaben | |
| 4. | Fancorgel ® A | 4.00 | Sodium Acrylates | Emulsifier/ |
| | | | Copolymer, | Thickener/ |
| | | | Dimethiconol | Conditioner |
| | | | Meadowfoamate, | |
| | | | Mineral Oil, | |
| | | | PPG-1 Trideceth-6 | |
| 5. | Fancol ® VB | 5.00 | Limnanthes Alba | Conditioner |
| | | | (Meadowfoam) | |
| | | | Seed Oil, | |
| | | | Butyrospermum | |
| | | | Parkii (Shea Butter) | |
| | | | Extract | |
| 6. | Meadowestolide | 2.00 | Meadowfoam | Active Agent |
| | | | Estolide | |
| 7. | DMDM Hydantoin | 0.40 | DMDM Hydantoin | Preservative |

Preparative Procedure:
Ingredients 1., 2. and 3. are heated to 45° C. in a main vessel. Once uniform, ingredient 4. is added, and the ingredients are mixed well until the mixture thickens. Ingredients 5., 6. and 7. are added and mixed with the mixture until homogenous. The resulting mixture is cooled to 30° C.
Ingredients 4., 5. and 6. are commercially available from The Fanning Corporation (Chicago, Ill.).
   Characteristics:
pH: 6-7
Viscosity: 15,000-25,000 cps

### Example 11

Leave-On Bi-Layer Sunscreen

| Ingredient | | Weight % | INCI Name | Function |
|---|---|---|---|---|
| 1. | Deionized Water | q.s | Water | Diluent |
| 2. | Methylparaben, | 0.25 | Methylparaben, | Preservative |
| | Propylparaben | | Propylparaben | |
| 3. | FD&C Red 33 | 0.003 | Red 33 | Color |
| 4. | Fancol ® IH-CG | 25.0 | Isohexadecane | Emollient |
| 5. | Isopropyl Myrsitate | 14.00 | Isopropyl Myristate | Conditioner |
| 6. | Meadowestolide | 3.00 | Meadowfoam | Active Agent |
| | | | Estolide | |
| 7. | D&C Green #6 | 0.0006 | Green 6 | Color |
| 8. | Octyl | 7.50 | Octyl | Sunscreen |
| | Methoxycinnamate | | Methoxycinnamate | Agent |
| 9. | Tocopheryl Acetate | 0.05 | Tocopheryl Acetate | Antioxidant |

Preparative Procedure:
In a main tank, ingredients 2. and 3. are mixed in ingredient 1. until uniform. Once clear, the mixture is set aside. In a separate vessel, ingredients 4., 5., 6., 7., 8. and 9. are combined, and are mixed until uniform and homogeneous. When both tanks (phases) are ready, they are put into a packaging container in a ratio of 50:50.
Ingredients 4. and 6. are commercially available from The Fanning Corporation (Chicago, Ill.).
   Appearance:
Clear Distinct Bi-layers of Light Green and Red Liquids

### Example 12

Moisturizing Skin Cream

| Ingredient | | Weight % | INCI Name | Function |
|---|---|---|---|---|
| 1. | D.I. Water | q.s. | Water | Diluent |
| 2. | Methylparaben, | 0.25 | Methylparaben, | Preservative |
| | Propylparaben | | Propylparaben | |
| 3. | Glycerin, 99% | 3.00 | Glycerin | Humectant |
| 4. | Fancor ® Uni- | 5.00 | Cetearyl Alcohol, | Emulsifier/ |
| | Embase | | Polysorbate 65, | Thickener |
| | | | Dimethicone PEG-8 | |
| | | | Meadowfoamate, | |
| | | | Meadowfoam | |
| | | | Amidopropyldimethyl | |
| | | | Betaine | |
| 5. | Cetearyl Alcohol | 0.50 | Cetearyl Alcohol | Thickener |
| 6. | Mineral Oil, | 10.00 | Mineral Oil | Emollient |
| | (70VS) | | | |
| 7. | Meadowestolide | 2.00 | Meadowfoam | Active Agent |
| | | | Estolide | |
| 8. | DMDM | 0.40 | DMDM Hydantoin | Preservative |
| | Hydantoin | | | |
| 9. | Matricaria Extract | 0.15 | Matricaria Extract | Botanical |

Preparative Procedure:
The ingredients are heated separately to 80° C., and then combined and mixed well with good shear. The mixture is dearated and cooled to 55° C. with continued agitation. Ingredients 4. and 7. are commercially available from The Fanning Corporation (Chicago, Ill.).
   Appearance/Characteristics:
White cream
pH: 4.25-5.25
Viscosity: 10,000-15,000 cps

### Example 13

Skin Moisturizing Lotion

| Ingredient | | Weight % | INCI Name | Function |
|---|---|---|---|---|
| 1. | Deionized Water | q.s. | Water | Diluent |
| 2. | Propylene Glycol, | 5.00 | Propylene Glycol | Humectant |
| | USP | | | |
| 3. | Methylparaben | 0.25 | Methylparaben | Preservative |
| 4. | Mineral Oil | 7.50 | Mineral Oil, | Emollient |
| | | | Propylparaben | |
| 5. | Fancor ® Uni- | 4.50 | Cetearyl Alcohol, | Emulsifier/ |
| | Embase | | Polysorbate 65, | Conditioner |
| | | | Dimethicone PEG-8 | |
| | | | Meadowfoamate, | |
| | | | Meadowfoam | |
| | | | Amidopropyldimethyl | |
| | | | Betaine | |
| 6. | Cetearyl Alcohol | 0.75 | Cetearyl Alcohol | Thickener |
| 7. | Meadowestolide | 2.00 | Meadowfoam | Active Agent |
| | | | Estolide | |
| 8. | Fancol ® VB | 2.50 | Limnanthes Alba | Emollient/ |
| | | | (Meadowfoam) | Conditioner |
| | | | Seed Oil, | |
| | | | Butyrospermum | |
| | | | ParkiiI (Shea Butter) | |
| | | | Extract | |
| 9. | Camelia Sinersis | 0.10 | Camelia Sinersis | Stabilizer |
| | Extract | | Extract | |
| 10. | DMDM | 0.40 | DMDM Hydantoin | Preservative |
| | Hydantoin | | | |

Preparative Procedure:
Ingredients 1., 2. and 3. were heated to 75° C. in a main vessel. Separately, ingredients 4., 5., 6., 7. and 8. were heated to 80° C., and added to the water phase. The ingredients were mixed well for 15 minutes using slow agitation and cooled to 45° C. Ingredients 9. and 10. were added and mixed until the mixture was homogenous. The mixture was cooled to 30° C. and then poured into containers.
Ingredients 5., 7. and 8. are commercially available from The Fanning Corporation (Chicago, Ill.).
   Appearance/Characteristics:
White Lotion
pH: 4.50-5.50
Viscosity: 5,000-8000 cps @ 25 C.

### Example 14

Skin Moisturizing Lotion

| | | Weight | | |
|---|---|---|---|---|
| Ingredient | | % | INCI Name | Function |
| 1. | Deionized Water | q.s | Water | Diluent |
| 2. | Propylene Glycol, | 5.00 | Propylene Glycol | Humectant |
| | USP | | | |
| 3. | Fanwax ® SEW-P | 4.50 | Emulsifying Wax, NF | Emulsifier |
| 4. | Cetearyl Alcohol | 1.00 | Cetearyl Alcohol | Conditioner |
| 5. | Mineral Oil, 70vs | 7.50 | Mineral Oil | Emollient |
| 6. | Meadowestolide | 2.50 | Meadowfoam Estolide | Active Agent |
| 7. | Phenoxyethanol, | 1.00 | Phenoxyethanol, | Preservative |
| | Methylparaben, | | Methylparaben, | |
| | Ethylparaben, | | Ethylparaben, | |
| | Propylparaben, and | | Propylparaben, and | |
| | Butylparaben | | Butylparaben | |
| 8. | Perfume | 0.25 | Fragrance | Aroma |

Preparative Procedure:
Ingredients 1. and 2. were heated to 75° C. Separately, ingredients 3., 4., 5., 6. and 7. were heated to 75° C. These ingredients were combined with the water phase slowly, and the mixture was mixed well and cooled to 45° C. Ingredient 8. was added, and cooling was continued to 30° C. with good agitation.
Ingredients 3. and 6. are commercially available from The Fanning Corporation (Chicago, Ill.).
   Appearance/Characteristics:
Off white, thick lotion
pH: 5-6 @ 25° C.
Viscosity: 7,000-10,000 cps. @ 25° C.

### Example 15

Skin Softening Cream

| | | Weight | | |
|---|---|---|---|---|
| Ingredient | | % | INCI Name | Function |
| 1. | Deionized Water | q.s | Water | Diluent |
| 2. | Glycerine, 99.0% | 4.00 | Glycerin | Humectant |
| 3. | Fanwax ® Sew-P | 4.50 | Emulsifying Wax, NF | Emulsifier |
| 4. | Cetearyl Alcohol | 2.00 | Cetearyl Alcohol | Conditioner |
| 5. | Mineral Oil, 70vs | 8.00 | Mineral Oil | Emollient |
| 6. | Fancor ® Lanolin, | 3.00 | Lanolin | Conditioner/ |
| | USP | | | Emollient |
| 7. | Meadowestolide | 2.00 | Meadowfoam | Active Agent |
| | | | Estolide | |
| 8. | Phenoxyethanol, | 1.00 | Phenoxyethanol, | Preservative |
| | Methylparaben, | | Methylparaben, | |
| | Ethylparaben, | | Ethylparaben, | |
| | Propylparaben, and | | Propylparaben, and | |
| | Butylparaben | | Butylparaben | |
| 9. | Fragrance | 0.25 | Fragrance | Aroma |

Preparative Procedure:
Ingredients 1. and 2. are heated to 75° C. Separately, ingredients 3., 4., 5., 6. and 7. are heated to 75° C. These ingredients are combined with the water phase slowly and the mixture is mixed well and cooled to 45° C. Ingredients 8. and 9. are added, and the mixture is cooled to 30° C. with good agitation.
Ingredients 3., 6. and 7. are commercially available from The Fanning Corporation (Chicago, Ill.).
   Appearance/Characteristics:
Off white, thick cream
pH: 5-6 @ 25° C.
Viscosity: 20,000-25,000 cps. @ 25° C.

### Example 16

Body Oil

| Ingredient | | Weight % | INCI Name | Function |
|---|---|---|---|---|
| 1. | EmCon ® SAF | 93.47 | Safflower Oil | Skin |
| | | | | Conditioner |
| 2. | Fancol ® CH | 0.03 | Cholesterol | Skin Toning |
| | | | | Conditioner |
| 3. | Meadowestolide | 1.00 | Meadowfoam | Active Agent |
| | | | Estolide | |
| 4. | Fanoliv ® ActivE | 3.00 | Olive (Olea Europa) | Emollient/ |
| | | | Oil Unsaponifiables | Anti-oxidant |
| 5. | Fancol ® Karite | 2.00 | Butyrospermum Parkii | Moisturizer |
| | Extract | | (Shea Butter) Fruit | |
| 6. | Phenoxyethanol | 0.40 | Phenoxyethanol | Preservative |
| 7. | Fragrance | 0.10 | Fragrance | Perfume |

Preparative Procedure:
Ingredient 1. is heated to 60° C. Ingredients 2. and 3. are mixed in together until they are well dissolved and uniform. The mixture is cooled to 45° C. Ingredients 4., 5., 6. and 7. are added to the mixture and mixed until homogenous, which results in a very clear oily liquid.
Ingredients 1., 2., 3., 4. and 5. are commercially available from The Fanning Corporation (Chicago, Ill.).
   Appearance:
Sparkling clear, slightly yellow oily liquid

While the present invention has been described herein with specificity, and with reference to certain preferred embodiments thereof, those of ordinary skill in the art will recognize numerous variations, modifications and substitutions of that which has been described which can be made, and which are within the scope and spirit of the invention.

It is intended that all of these modifications and variations be within the scope of the present invention as described and claimed herein, and that the invention be limited only by the scope of the claims which follow, and that such claims be interpreted as broadly as is reasonable.

Throughout this document, various books, patents, journal articles, web sites and other publications have been cited. The entireties of each of these books, patents, journal articles, web sites and other publications are hereby incorporated by reference herein.

What is claimed is:

1. A method for enhancing a morphology, tone, texture or appearance, or a combination thereof, of a mammal's skin comprising topically applying to the skin on a regular basis at least three applications of a composition including:
(a) a Meadowestolide in an amount that is effective for enhancing the morphology, tone, texture or appearance, or a combination thereof, of the skin, wherein the Meadowestolide has the structure: 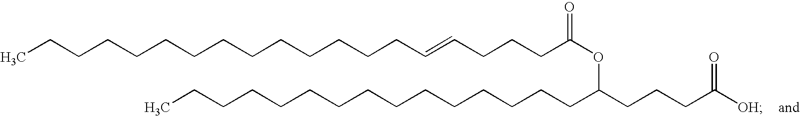
(b) a cosmetically acceptable base in an amount that is effective for acting as a carrier vehicle for the Meadowestolide;
wherein the amount of the composition that is applied to the skin is an amount that is effective for enhancing the morphology, tone, texture or appearance, or a combination thereof, of the mammal's skin;
wherein the pH of the composition ranges from about 2 to about 11; and
wherein the morphology, tone, texture or appearance, or a combination thereof, of the mammal's skin is enhanced.

2. The method of claim 1 wherein the amount of the Meadowestolide ranges from about 0.1 to about 20 weight percent of the total weight of the composition.

3. The method of claim 2 wherein the amount of the Meadowestolide ranges from about 1 to about 5 weight percent of the total weight of the composition.

4. The method of claim 3 wherein the amount of the Meadowestolide ranges from about 1.5 to about 4.5 weight percent of the total weight of the composition.

5. The method of claim 4 wherein the amount of the Meadowestolide ranges from about 2.0 to about 4.0 weight percent of the total weight of the composition.

6. The method of claim 5 wherein the amount of the Meadowestolide ranges from about 2.5 to about 3.5 weight percent of the total weight of the composition.

7. The method of claim 2 wherein the amount of the Meadowestolide ranges from about 0.1 to about 4 weight percent of the total weight of the composition.

8. The method of claim 2 wherein the amount of the Meadowestolide ranges from about 6 to about 20 weight percent of the total weight of the composition.

9. The method of claim 1 wherein the composition is a leave-on type composition.

10. The method of claim 1 wherein the cosmetically acceptable base is a cream base that contains the following ingredients, and weight percents thereof:

| | |
|---|---|
| Deionized water | 87.0-90.0% |
| Self-emulsifying wax N.F. | 7.75% |
| Methylparaben/propylparaben | 0.25% |

11. The method of claim 2 wherein the composition is a leave-on type composition and the cosmetically acceptable base is a cream base that contains the following ingredients, and weight percents thereof:

| | |
|---|---|
| Deionized water | 87.0-90.0% |
| Self-emulsifying wax N.F. | 7.75% |
| Methylparaben/propylparaben | 0.25% |

12. The method of claim 1 wherein the composition is a rinse-off type composition, and wherein the composition is not rinsed off of, or otherwise removed from, the skin after an application of the composition to the skin for a period of time that is sufficient for conditioning the skin.

13. The method of claim 12 wherein the composition is not rinsed off of, or otherwise removed from, the skin after an application of the composition to the skin for a period of at least about six minutes after such application.

14. The method of claim 1 wherein the composition contains the following ingredients, and weight percents thereof:

| | |
|---|---|
| Deionized water | 90.0% |
| Self-emulsifying wax N.F. | 7.75% |
| Methylparaben/propylparaben | 0.25% |
| Meadowestolide | 2.0%. |

15. The method of claim 1 wherein the amount of the composition that is applied to the skin per each application ranges from about 1 to about 20 g or ml.

16. The method of claim 15 wherein the amount of the composition that is applied to the skin per each application ranges from about 2 to about 12 g or ml.

17. The method of claim 16 wherein the amount of the composition that is applied to the skin per each application ranges from about 3 to about 8 g or ml.

18. The method of claim 17 wherein the amount of the composition that is applied to the skin per each application ranges from about 4 to about 6 g or ml.

19. The method of claim 18 wherein the amount of the composition that is applied to the skin per each application is about 5 g or ml.

20. The method of claim 14 wherein the amount of the composition that is applied to the skin per each application ranges from about 1 to about 20 g or ml.

21. The method of claim 20 wherein the amount of the composition that is applied to the skin per each application ranges from about 2 to about 12 g or ml.

22. The method of claim 1 wherein three or more applications of the composition are applied to the skin of the mammal over a period of time ranging from about three hours to about three days.

23. The method of claim 1 wherein four or more applications of the composition are applied to the skin of the mammal over a period of time ranging from about four hours to about four days.

24. The method of claim 1 wherein five or more applications of the composition are applied to the skin of the mammal over a period of time ranging from about five hours to about five days.

25. The method of claim 1 wherein six or more applications of the composition are applied to the skin of the mammal over a period of time ranging from about six hours to about six days.

26. The method of claim 1 wherein seven or more applications of the composition are applied to the skin of the mammal over a period of time ranging from about seven hours to about seven days.

27. The method of claim 1 wherein eight or more applications of the composition are applied to the skin of the mammal over a period of time ranging from about eight hours to about eight days.

28. The method of claim 1 wherein nine or more applications of the composition are applied to the skin of the mammal over a period of time ranging from about nine hours to about nine days.

29. The method of claim 1 wherein ten or more applications of the composition are applied to the skin of the mammal over a period of time ranging from about ten hours to about ten days.

30. The method of claim 1 wherein eleven or more applications of the composition are applied to the skin of the mammal over a period of time ranging from about eleven hours to about eleven days.

31. The method of claim 1 wherein twelve or more applications of the composition are applied to the skin of the mammal over a period of time ranging from about twelve hours to about twelve days.

32. The method of claim 1 wherein thirteen or more applications of the composition are applied to the skin of the mammal over a period of time ranging from about thirteen hours to about thirteen days.

33. The method of claim 1 wherein fourteen or more applications of the composition are applied to the skin of the mammal over a period of time ranging from about fourteen hours to about fourteen days.

34. The method of claim 1 wherein fifteen or more applications of the composition are applied to the skin of the mammal over a period of time ranging from about fifteen hours to about fifteen days.

35. The method of claim 1 wherein sixteen or more applications of the composition are applied to the skin of the mammal over a period of time ranging from about sixteen hours to about sixteen days.

36. The method of claim 1 wherein seventeen or more applications of the composition are applied to the skin of the mammal over a period of time ranging from about seventeen hours to about seventeen days.

37. The method of claim 1 wherein eighteen or more applications of the composition are applied to the skin of the mammal over a period of time ranging from about eighteen hours to about eighteen days.

38. The method of claim 1 wherein nineteen or more applications of the composition are applied to the skin of the mammal over a period of time ranging from about nineteen hours to about nineteen days.

39. The method of claim 1 wherein twenty or more applications of the composition are applied to the skin of the mammal over a period of time ranging from about twenty hours to about twenty days.

40. The method of claim 1 wherein twenty one or more applications of the composition are applied to the skin of the mammal over a period of time ranging from about twenty one hours to about twenty one days.

41. The method of claim 1 wherein twenty two or more applications of the composition are applied to the skin of the mammal over a period of time ranging from about twenty two hours to about twenty two days.

42. The method of claim 1 wherein twenty three or more applications of the composition are applied to the skin of the mammal over a period of time ranging from about twenty three hours to about twenty three days.

43. The method of claim 1 wherein twenty four or more applications of the composition are applied to the skin of the mammal over a period of time ranging from about twenty four hours to about twenty four days.

44. The method of claim 1 wherein from about twenty five to about sixty applications of the composition are applied to the skin of the mammal over a period of time ranging from about twenty five hours to about fifty days.

45. The method of claim 1 wherein from about sixty one to about one hundred and twenty applications of the composition are applied to the skin of the mammal over a period of time ranging from about sixty one hours to about sixty days.

46. The method of claim 1 wherein from about one hundred and twenty one to about two hundred and forty applications of the composition are applied to the skin of the mammal over a period of time ranging from about one hundred and twenty one hours to about eighty days.

47. The method of claim 1 wherein the composition does not include meadowfoam oil or any components of meadowfoam oil.

48. The method of claim 1 wherein the composition does not include a hydroxyphenyltriazine compound or an HDI/trimethylol hexyl-lactone crosspolymer.

49. The method of claim 1 wherein the composition does not include bioactive glass, or a composition derived from an aqueous extract of bioactive glass.

50. The method of claim 1 wherein the composition does not include cystine, a cystine derivative or a salt thereof.

51. A method for enhancing a morphology, tone, texture or appearance, or a combination thereof, of a mammal's skin comprising topically applying to the skin on a regular basis at least three applications of a composition consisting essentially of:
(a) a Meadowestolide in an amount that is effective for enhancing the morphology, tone, texture or appearance, or a combination thereof, of the skin, wherein the Meadowestolide has the structure: 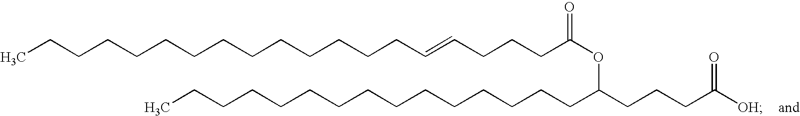
(b) a cosmetically acceptable base in an amount that is effective for acting as a carrier vehicle for the Meadowestolide;
wherein the amount of the composition that is applied to the skin is an amount that is effective for enhancing the morphology, tone, texture or appearance, or a combination thereof, of the mammal's skin;
wherein the pH of the composition ranges from about 2 to about 11; and
wherein the morphology, tone, texture or appearance, or a combination thereof, of the mammal's skin is enhanced.
